SEQUENCE LISTING (47 sequences)

SEQ ID NO: 1 - DNA, 20 bp, artificial sequence
  guide RNA cDNA
attcttcatccatatatcgc

SEQ ID NO: 2 - DNA, 20 bp, artificial sequence
  guide RNA cDNA
ggcagtgctcaccgagctgc

SEQ ID NO: 3 - DNA, 20 bp, artificial sequence
  guide RNA cDNA
aagatcacaagttatactgg

SEQ ID NO: 4 - DNA, 20 bp, artificial sequence
  guide RNA cDNA
tgcctatccaaaccgcccaa

SEQ ID NO: 5 - DNA, 20 bp, artificial sequence
  guide RNA cDNA
cagaactccaaaagaccaga

SEQ ID NO: 6 - DNA, 20 bp, artificial sequence
  guide RNA cDNA
aggactaagacatggcacaa

SEQ ID NO: 7 - DNA, 20 bp, artificial sequence
  guide RNA cDNA
ccacactggcgtagtaaggg

SEQ ID NO: 8 - DNA, 20 bp, artificial sequence
  primer
gttgagaatgcagggtccga

SEQ ID NO: 9 - DNA, 20 bp, artificial sequence
  primer
tgaccttgtccgaagtgacc

SEQ ID NO: 10 - DNA, 20 bp, artificial sequence
  primer
taggacccatcgcttgtctt

SEQ ID NO: 11 - DNA, 20 bp, artificial sequence
  primer
acggccaattcattcacttc

SEQ ID NO: 12 - DNA, 20 bp, artificial sequence
  primer
actctttggtggtggactgg

SEQ ID NO: 13 - DNA, 20 bp, artificial sequence
  primer
atccatcttgctccaacacc

SEQ ID NO: 14 - DNA, 20 bp, artificial sequence
  primer
tcgcggttgttacttgactg

SEQ ID NO: 15 - DNA, 22 bp, artificial sequence
  primer
cctctgctttctagtgatgccg

SEQ ID NO: 16 - DNA, 22 bp, artificial sequence
  primer
cgtaaacacggtcttgctcctg

SEQ ID NO: 17 - DNA, 22 bp, artificial sequence
  primer
caacataggcggtggtggcaat

SEQ ID NO: 18 - DNA, 22 bp, artificial sequence
  primer
gttgatgctccaggaacactgg

SEQ ID NO: 19 - DNA, 22 bp, artificial sequence
  primer
ggtgcctatgtctcagcctctt

SEQ ID NO: 20 - DNA, 23 bp, artificial sequence
  primer
gccatagaactgatgagagggag

SEQ ID NO: 21 - DNA, 22 bp, artificial sequence
  primer
gaggtggagtttgatgtgctgc

SEQ ID NO: 22 - DNA, 22 bp, artificial sequence
  primer
gtgaagcaggtagagaactcgc

SEQ ID NO: 23 - DNA, 22 bp, artificial sequence
  primer
ctgtgctgacctcagagaagtc

SEQ ID NO: 24 - DNA, 22 bp, artificial sequence
  primer
tgcccttgagtgtggtgccttt

SEQ ID NO: 25 - DNA, 23 bp, artificial sequence
  primer
gacttccgacatcaagaggtctg

SEQ ID NO: 26 - DNA, 22 bp, artificial sequence
  primer
atccaggtctgagcctcctttg

SEQ ID NO: 27 - DNA, 22 bp, artificial sequence
  primer
gagtctcatccgcctggtcaaa

SEQ ID NO: 28 - DNA, 22 bp, artificial sequence
  primer
ccaggcatagacagtgagtagc

SEQ ID NO: 29 - DNA, 22 bp, artificial sequence
  primer
ttctctgccagcttcggaaagc

SEQ ID NO: 30 - DNA, 22 bp, artificial sequence
  primer
ctctgaaggcagacttgtgacc

SEQ ID NO: 31 - DNA, 22 bp, artificial sequence
  primer
atgcactcttctgagctggtgg

SEQ ID NO: 32 - DNA, 22 bp, artificial sequence
  primer
tcagatccaggctatccacgtg

SEQ ID NO: 33 - DNA, 22 bp, artificial sequence
  primer
cttcactgggacaggcattgca

SEQ ID NO: 34 - DNA, 22 bp, artificial sequence
  primer
cctgctgattggagtgtggcta

SEQ ID NO: 35 - DNA, 22 bp, artificial sequence
  primer
gcagagaatgctatcctggagg

SEQ ID NO: 36 - DNA, 22 bp, artificial sequence
  primer
cagcggattcatctgcttcgtg

SEQ ID NO: 37 - DNA, 22 bp, artificial sequence
  primer
ccaatgtcaggtgtgaaccagg

SEQ ID NO: 38 - DNA, 22 bp, artificial sequence
  primer
agtctgggttgagtggctttcc

SEQ ID NO: 39 - DNA, 20 bp, artificial sequence
  primer
tgcaaatgccagccctaaga

SEQ ID NO: 40 - DNA, 20 bp, artificial sequence
  primer
ccattttccactccccacca

SEQ ID NO: 41 - DNA, 23 bp, artificial sequence
  primer
caggaactgatagtaattgcccg

SEQ ID NO: 42 - DNA, 22 bp, artificial sequence
  primer
cttcaacttgcgagcagcacga

SEQ ID NO: 43 - DNA, 19 bp, artificial sequence
  primer
ctgtccctgtatgcctctg

SEQ ID NO: 44 - DNA, 18 bp, artificial sequence
  primer
atgtcacgcacgatttcc

SEQ ID NO: 45 - DNA, 22 bp, artificial sequence
  primer
caccattggcaatgagcggttc

SEQ ID NO: 46 - DNA, 22 bp, artificial sequence
  primer
aggtctttgcggatgtccacgt

SEQ ID NO: 47 - protein, 8 aa, artificial sequence
  synthetic peptide antigen
SIINFEKL

(19) United States
(12) Patent Application Publication
(10) Pub. No.: US 2022/0356264 A1
(43) Pub. Date: Nov. 10, 2022

(54) INHIBITION OF ATF7IP AND SETDB1 FOR CANCER TREATMENT

(71) Applicant: New York University, New York, NY (US)

(72) Inventors: Kwok-Kin WONG, Arlington, MA (US); Hai HU, New York, NY (US); Fei LI, New York, NY (US)

(21) Appl. No.: 17/738,805

(22) Filed: May 6, 2022

Related U.S. Application Data
(60) Provisional application No. 63/184,872, filed on May 6, 2021.

(51) Int. Cl. C07K 16/30; A61P 35/00; C07K 16/28

(52) U.S. Cl. CPC C07K 16/30; A61P 35/00; C07K 16/2827; A61K 2039/505

(57) ABSTRACT

Provided are methods for cancer therapy. The method comprises inhibiting the expression and/or activity of ATF7IP and/or SETDB1 in cancer cells. Also provided is a method for identifying individuals for cancer therapy that includes using ATF7IP and/or SETDB1 inhibition.

### CROSS REFERENCE TO RELATED APPLICATIONS

This application claims priority to U.S. application No. 63/184,872, filed May 6, 2021, the entire disclosure of which is incorporated herein by reference.

### SEQUENCE LISTING

The instant application contains a Sequence Listing which has been submitted electronically in ASCII format and is hereby incorporated by reference in its entirety. Said ASCII file, created on May 5, 2022, is named NYU_00513_ST25.txt, and is 8,725 bytes in size.

### BACKGROUND OF THE DISCLOSURE

Immune cells play a key role in preventing cancer development in immunocompetent hosts via immune surveillance. However, cancer cells are capable of escaping the surveillance from the immune system during the cancer immunoediting process (Schreiber et al., 2011). Although considerable knowledge has accumulated on how tumors avoid immune surveillance, the development of effective therapies to overcome tumor immune evasion remains a challenge.

Moreover, cancer cells can hijack immune checkpoint pathways to suppress T cell function and escape immune surveillance. Therefore, immune checkpoint inhibitors (“ICIs”) have been developed to restore and maintain the activity of tumor specific T cells. To date, ICIs have displayed some success in treating multiple cancers, including metastatic melanoma, advanced non-small cell lung cancer and colorectal cancer. Unfortunately, a significant proportion of patients that show initial response eventually acquire resistance Thus, searching for therapeutic targets that can promote tumor antigen expression and/or boost anti-tumor immunity is urgently needed for cancer immunotherapy.

### SUMMARY OF THE DISCLOSURE

In this disclosure we identified factors ATF7IP or SETDB1 that can inhibit the growth of cancer cells and/or enhance anti-tumor response. An immune escaped tumor model was established with silenced antigen expression and an epigenetic CRISPR screen was performed. We observed that loss of the chromatin modifiers ATF7IP or SETDB1 in tumor cells restored tumor antigen expression. ATF7IP or SETDB1 inhibition further augmented tumor immunogenicity at least in part by elevating endogenous retroviral antigens expression and RNA intron retention. We observed elevated type I interferon response, increased T cell infiltration, and ultimately stimulated anti-tumor immunity. Based at least in part on these results, the present disclosure provides compositions and methods for targeting ATF7IP or SETDB1 in cancer immunotherapy.

In an aspect, this disclosure provides a method of inhibiting the growth of cancer cells and/or enhancing anti-tumor immune response comprising inhibiting the expression or activity of ATF7IP and/or SETDB1. These factors may act as epigenetic factors. The activity of ATF7IP and/or SETDB1 may be inhibited at any level. For example, expression and/or activity of ATF7IP and/or SETDB1 may be inhibited by CRISPR or RNAi technologies or by using specific inhibitors of these factors. In some embodiments, this disclosure provides a method for enhancing the efficacy of cancer immune therapy by administering the therapy in conjunction with inhibiting the expression or activity of ATF7IP and/or SETDB1.

In an aspect, this disclosure provides a method of identifying patients who are suited for immune checkpoint inhibition (ICI) therapy. The method comprises screening individuals who are diagnosed with cancer for the expression/activity of ATF7IP and/or SETDB1, and if the levels of expression or activity of these factors is found to be low (compared to a reference), then considering the ICI therapy to be suitable for the individual, and if the level of expression or activity of these factors is found to be high then identifying the individuals to not be suitable for ICI therapy. In various embodiments, patients who have high level of expression or activity of ATF7IP and/or SETDB1 can be subjected to lowering the expression or activity of these factors prior to initiation of ICI.

### BRIEF DESCRIPTION OF THE FIGURES AND TABLES

The patent or application file contains at least one drawing executed in color. Copies of this patent or patent application publication with color drawing(s) will be provided by the Office upon request and payment of the necessary fee.

FIG. 1. The characteristics of immune escaped tumor model. A, Strategy of establishing immune escaped cell line. KP: KrasG12D/P53−/− murine lung cancer cells; KP-SQ: KP cells expressing SQ immunogen; KP-C6: the single clone of KP-SQ cells; KP-IE1: 1st generation of immune escaped cell line; KP-IE2: 2nd generation of immune escaped cell line. B, FACS plot of H-2Kb bound SIINFEKL (SEQ ID NO:47), major histocompatibility complex (MHC) H-2 and PD-L1. C, RT-PCR analysis of SQ transcripts in different cell lines. D, RT-PCR analysis of genomic SQ copy number in different cell lines. E, Animation of CpG site methylation on SQ promoter. F, Quantification of methylated CpG sites on SQ promoter. G, DNA accessibility of SQ region evaluated by ATAC seq. H, Quantification of DNA accessibility of SQ region. All data are mean±SEM. *p<0.05.

FIG. 2. Epigenetic CRISPR screen identifies Atf7ip as a target for regulating SQ expression and presentation. A, Strategy of epigenetic CRISPR screen to identify epigenetic factors of antigen expression and presentation. B, Volcano plot illustrating genes whose inhibition can enhance (red) or inhibit (blue) H-2Kb bound SIINFEKL (SEQ ID NO:47) presentation. C, Illustration of the top 10 candidates from (B). D, Normalized read counts of Atf7ip sgRNAs in SIINFEKL (SEQ ID NO:47) 15% high and 15% low populations. E, RT-PCR analysis of SQ transcripts. F, FACS plot of H-2Kb bound SIINFEKL (SEQ ID NO:47) presentation after knocking out Atf7ip with 2 different sgRNAs. G, FACS analysis of H-2Kb bound SIINFEKL (SEQ ID NO:47) presentation. H, Animation of CpG site methylation on SQ promoter, KP-IE2-C1 (the single clone of KP-IE2). I, Quantification of methylated CpG sites on SQ promoter. J, Representative MRI scans (1 of 24 scanned images of each mouse) showing mouse lung tumors 4 weeks after control and Atf7ip deficient KPIE2 cells transplant through tail vein injection in B6/J immunocompetent mice. K, Quantification of lung tumors volume in (J), L, Survival curves for each group in the study (J) (sglacZ n=9, sgAtf7ip_1 n=8, sgAtf7ip_2 n=8). M, Survival curves for the immunocompromised nude mice (NU/NU) after being transplanted with control and Atf7ip deficient KPIE2 cells via tail vein injection (sglacZ n=3, sgAtf7ip_1 n=3, sgAtf7ip_2 n=3). All data are mean±SEM. *p<0.05, **p<0.01, ***p<0.001, ****p<0.0001.

FIG. 3. Atf7ip inhibition stimulates anti-tumor immunity in vivo. A-B, Tumor growth curves of MC3 8 tumors with or without Atf7ip deficiency in (NU/NU) mice and (B6/J) mice (NU/NU: sglacZ n=8 sgAtf7ip_1 n=6 sgAtf7ip_2 n=6, B6/J: sglacZ n=16 sgAtf7ip_1 n=12 sgAtf7ip_2 n=12). C, Normalized tumor volume of B6/J mice at day18. D, CD3 Immunohistochemistry staining of tumor samples at day19. E, Quantification of CD3⁺ cells in (D). F, Bar graft of CD8⁺ T cells percentage in CD45⁺ cells in the tumor samples at day19. G-H, Tumor growth curves of YUMM1.7 tumors with or without Atf7ip deficiency in (NU/NU) mice and (B6/J) mice (NU/NU: sglacZ n=6 sgAtf7ip_1 n=6 sgAtf7ip_2 n=6, B6/J: sglacZ n=12 sgAtf7ip_1 n=12 sgAtf7ip_2 n=12). I, Volcano plot illustrating retroelements that are upregulated (red) in Atf7ip deficient MC38 cells. J, Quantification of the number of ERVs in (G) (adj. p-value<0.05, fold change>2). K, RT-PCR analysis of endogenous retroviral tumor associated antigens p15E and gp70. L-M, Enrichment of genes associated with INTERFERON_ALPHA_RESPONSE (L) and INTERFERON_GAMMA_RESPONSE (M). N, RT-PCR analysis of interferon stimulated genes. O, Western blot of ERVs sensors and interferon signaling pathway components. P, RT-PCR analysis of Irf7 and Irf9. Q, Western blot of ERVs sensors and interferon signaling pathway components. R, Enrichment of cancer testis antigen genes. All data are mean±SEM. *p<0.05, **p<0.01, ***p<0.001, ****p<0.0001.

FIG. 4. Atf7ip and Setdb1 works in a complex to regulate SQ expression and presentation. A, Strategy to identify Atf7ip binding partners whose inhibition promotes SQ transcription and H-2 mediated presentation. B, Co-immunoprecipitation of Atf7ip and Setdb1. C, RT-PCR analysis of SQ transcripts. D, FACS analysis of SIINFEKL (SEQ ID NO:47) presentation. E, Quantification of SIINFEKL (SEQ ID NO:47) presentation in (D). F, Survival curve for the B6/J mice inoculated with wild type or Setdb1 deficient KPIE2 cells via tail vein injection (sglacZ n=9, sgSetdb1_1 n=8, sgSetdb1_2 n=8). G, Survival curve for the NU/NU mice inoculated with wild type or Setdb1 deficient KPIE2 cells via tail vein injection (sglacZ n=3, sgSetdb1_1 n=3, sgSetdb1_2 n=3). H-I, RT-PCR analysis of SQ transcripts after inhibiting Setdb1 in Atf7ip knocked-out KP-IE2 cells. J-K, FACS analysis of SIINFEKL (SEQ ID NO:47) presentation after inhibiting Setdb1 in Atf7ip knocked-out KP-IE2 cells. L-M, RT-PCR analysis of SQ transcripts after inhibiting Atf7ip in Setdb1 knocked-out KP-IE2 cells. N-O, FACS analysis of SIINFEKL (SEQ ID NO:47) presentation after inhibiting Atf7ip in Setdb1 knocked-out KP-IE2 cells. P-Q, Western blot analysis of Atf7ip and Setdb1 after depleting Setdb1 or Atf7ip in KP-IE2 cells (P) and MC38 cells (Q). R-S, Western blot analysis of Atf7ip and Setdb1 after overexpressing Atf7ip or Setdb1 or both Atf7ip and Setdb1 in KP-IE2 cells (R) and MC38 cells (S). PCDH is empty vector. All data are mean±SEM. ****p<0.0001.

FIG. 5. Setdb1 inhibition stimulates anti-tumor immunity in vivo. A-C, Tumor growth curves of MC38 tumors with or without Setdb1 deficiency in (NU/NU) mice and (B6/J) mice (NU/NU: sglacZ n=8 sgSetdb1_1 n=6 sgSetdb1_2 n=6 sgSetdb1_3 n=6, B6/J: sglacZ n=8 sgSetdb1_1 n=8 sgSetdb1_2 n=8 sgSetdb1_3 n=8). D, Normalized tumor volume of B6/J mice at day18. E, CD3 immunohistochemistry staining of tumor samples at day19. F, Quantification of CD3⁺ cells in (E). G-H, Bar graft of CD4⁺ (G) and CD8⁺ (H) T cells percentage in CD45⁺ cells in the tumor samples at day19. I-J, Tumor growth curves of YUMM1.7 tumors with or without Setdb1 deficiency in nude (NU/NU) mice and B6 wild type (B6/J) mice (NU/NU: sglacZ n=6 sgSetdb1_1 n=6 sgSetdb1_3 n=6, B6/J: sglacZ n=12 sgSetdb1_1 n=12 sgSetdb1_3 n=12). K, Volcano plot illustrating retroelements that are upregulated (red) in Setdb1 deficient MC38 cells. L, Quantification of the number of ERVs in (K) (adj. p-value<0.05, fold change>2). M, RT-PCR analysis of p15E and gp70. N-O, Enrichment of genes associated with INTERFERON_ALPHA_RESPONSE (N) and INTERFERON_GAMMA_RESPONSE (O). P, RT-PCR analysis of interferon stimulated genes. Q, Western blot analysis of ERVs sensors and interferon signaling pathway components. R, RT-PCR analysis of Irf7 and Irf9. S, Western blot of ERVs sensors and interferon signaling pathway components. T, Overlapped upregulated genes number in both Atf7ip KO and Setdb1 KO groups of MC38 cells. U, Enrichment of pathways associated with overlapping genes in (T) by Enrichr analysis tool. V, Enrichment of cancer testis antigen genes. All data are mean±SEM. *p<0.05, **p<0.01, ***p<0.001, ****p<0.0001.

FIG. 6. Chromatin remodeling enables ERVs upregulation upon Atf7ip or Setdb1 inhibition. A, Volcano plot illustrating H3K9 trimethylation of retroelements in MC38 cells with or without Atf7ip from differential H3K9me3 ChIP binding peaks analysis. B, Quantification of the number of ERVs with significant (adj. p-value<0.05) higher binding peaks in (A). C, Volcano plot illustrating H3K9 trimethylation of retroelements in MC38 cells with or without Setdb1 from differential H3K9me3 ChIP binding peaks analysis. D, Quantification of the number of ERVs with significant (adj. p-value<0.05) higher binding peaks in (C). E, Overlapped relative number of H3K9 demethylated ERVs in both Atf7ip KO and Setdb1 KO groups of MC38 cells. F, Intra-TAD interaction changes in Atf7ip deficient (sgAtf7ip) versus wild type (sglacZ) MC38 cells. Atf7ip inhibition is associated with increased intra-TAD interaction (red, 918) and decreased intra-TAD interaction (blue, 40). G, Intra-TAD interaction changes in Setdb1 deficient (sgSetdb1) versus wild type (sglacZ) MC38 cells. Setdb1 inhibition is associated with increased intra-TAD interaction (red, 786) and decreased intra-TAD interaction (blue, 94). H, CDF plot illustrating the distribution of ERVs expression associated by TADs that have increased interaction (red) and decreased interaction (blue) from wild type MC38 cells to Atf7ip deficient MC38 cells. I, CDF plot illustrating the distribution of ERVs expression associated by TADs that have increased interaction (red) and decreased interaction (blue) from wild type MC38 cells to Setdb1 deficient MC38 cells.

FIG. 7. Low ATFIP or SETDBJ expression is associated with hot tumor immune microenvironment in cancer patients. A, Bubble Pot showing multiple GSEA analysis of the indicated pathways in pan-cancer analysis of the TCGA dataset. NES>0: the gene set is enriched in ATF7IP low expressed cancer samples (bottom 25%) comparing with ATF7IP high expressed cancer samples (top 25%). B, Bubble Pot showing multiple GSEA analysis of the indicated pathways in pan-cancer analysis of the TCGA dataset. NES>0: the gene set is enriched in SETDB1 low expressed cancer samples (bottom 25%) comparing with SETDB1 high expressed cancer samples (top 25%).

FIG. 8. Atf7ip or Setdb1 overexpression results in tumor immune evasion. A, Western blot analysis of Atf7ip and Setdb1 after overexpressing Setdb1 or Atf7ip in YUMMER 1.7 cells. B, CCK-8 assay checking the proliferation of YUMMER 1.7 cells with Atf7ip or Setdb1 overexpression in culture. C, The formation of YUMMER 1.7 tumors with Atf7ip or Setdb1 overexpression in vivo allograft transplants. D, Working model for Atf7ip or Setdb1 deficiency in stimulating anti-tumor immunity.

FIG. 9. Immune escaped tumor in B6/J immunocompetent mice. (A) Magnetic resonance imaging scan of mouse lung tumor (indicated by red dash circle) from which the 1st generation of immune escaped cell line was established. (B) The number of mice developped with or without immune escaped tumor after 10 months immunoeditting.

FIG. 10. Atf7ip deficiency reverses SIINFEKL (SEQ ID NO:47) expression and presentation in immune escaped KP-LucOS model. (A) Simplified sequence Map for LucOS vector. (B) RT-PCR analysis of SIINFEKL (SEQ ID NO:47) transcripts in KP-LucOS parental cell line (P) and its derived immune escaped cell line IE. (C) RT-PCR analysis of SIINFEKL (SEQ ID NO:47) transcripts in immune escaped cell line IE and Atf7ip deficient IE (D) FACS analysis of SIINFEKL (SEQ ID NO:47) presentation in immune escaped cell line IE transfected with lacZ and Atf7ip sgRNAs. E, FACS analysis of SIINFEKL (SEQ ID NO:47) presentation in (D).

FIG. 11. Atf7ip inhibition has marginal effect on cell proliferation in vitro. (A) Western blot analysis of Atf7ip expression in KP_IE2 cells transfected with lacZ or Atf7ip sgRNAs. (B) CCK-8 assay checking the proliferation of KP_IE2 cells transfected with lacZ or Atf7ip sgRNAs in culture. (C) Western blot analysis Atf7ip expresson in MC38 cells transfected with lacZ or Atf7ip sgRNAs. (D) CCK-8 assay checking the proliferation of MC38 cells transfected with lacZ or Atf7ip sgRNAs in culture.

FIG. 12. Gating strategy for immune profile analyses in this disclosure.

FIG. 13. Atf7ip deficiency promotes the activation of infiltrated T-cells. (A) Representative flow cytometry analysis of CD69+ (T-cell activation marker) population of CD8+ T cells. (B) Scatter plot comparing the expression of CD69+ population of CD8+ T cells. (C) Representative flow cytometry analysis of CTLA4+ population of CD4+ T cells. (D) Scatter plots comparing the expression of CTLA4+ population of CD4+ T cells. (E) Representative flow cytometry analysis of CTLA4+ population of CD8+ T cells. (F) Scatter plots comparing the expression of CTLA4+ population of CD8+ T cells. (G) Representative flow cytometry analysis of T-bet+ population of CD4+ T cells. (H) Scatter plots comparing the expression of Tbet+ population of CD4+ T cells. (I) Representative flow cytometry analysis of T-bet+ population of CD8+ T cells. (J) Scatter plots comparing the expression of Tbet+ population of CD8+ T cells. For all flow cytometry experiments, the tumors from subcutaneous injection model were harvest and processed at day19 (sglacZ, n=6; sgAtf7ip, n=6). All data are mean±SEM. *, P<0.05; **, P<0.01.

FIG. 14. Atf7ip deficiency inhibited spliceosome activity and increased intron retention events. A-B, Enrichment of genes associated with SPLICEOSOME (A) and SPLICEOSOMAL_SNRNP_ASSEMBLY (B) in MC38 cell with Atf7ip inhibition. (C) Number of transcripts with retained intron in MC38 cell with or without Atf7ip deficiency. The plot represents the MATS analysis using 6 biological replicates. Only events that passed the statistics threshold FDR<0.05 and PSI>0.1 (10% of the transcripts of a given gene) are taking into consideration. (D-E) Enrichment of genes associated with SPLICEOSOME and SPLICEOSOMAL_SNRNP_ASSEMBLY in human LUAD tumors with low ATF7IP expression.

FIG. 15. Setdb1 inhibition promotes SQ presentation and transcription. (A) FACS analysis to check SIINFEKL (SEQ ID NO:47) presentation upon inhibiting top ranking Atf7ip binding partners. 3 different sgRNAs for each gene were used. (B) RT-PCRanalysis of SQ expression at RNA level. (C) Volcano plot illustrating genes whose knockout can enhance (red) or inhibit (blue) the SIINFEKL (SEQ ID NO:47) presentation. All data are mean±SEM. **p<0.01, ***p<0.001, ****p<0.0001

FIG. 16. The regulation of SQ presentation is independent of HUSH complex and other H3K9 methyltransferases. FACS analysis to check H2-Kb bound SIINFEKL (SEQ ID NO:47) presentation. 3 sgRNAs for each gene were used.

FIG. 17. Setdb1 deficiency promotes SIINFEKL (SEQ ID NO:47) expression in immune escaped KP-LucOS model. RT-PCR analysis of SIINFEKL (SEQ ID NO:47) transcripts in immune escaped cell line IE transfected with lacZ or Setdb1 sgRNAs.

FIG. 18. Generation of Setdb1 and Atf7ip depleted KP-IE2 cell clones. (A) Western blot analysis to check Setdb1 expression in single clones of KP-IE2. Setdb1 depleted cell clones (red color) were pooled together for in vivo and double genes deletion in vito experiments. (B) Western blot analysis to check Atf7ip expression in single clones of KP-IE2. Atf7ip depleted cell clones (red color) were pooled together for double genes deletion in vito experiments.

FIG. 19. Setdb1 inhibition stimulates anti-tumor immune response in KP lung adenocarcinoma model. (A) The abundance of Setdb1 sgRNAs in the tumor samples of IgG treated Rag1−/− mice and B6/J mice from our previous epigenomewide CRISPR screen. (B) Growth curve of KP tumors with or without Setdb1 knockdown. Doxycycline inducible shRNA system was used (shCtrl n=10 shSetdb1 n=10).

FIG. 20. Generation of Setdb1 depleted MC38 cell line. (A) Western blot analysis to check Setdb1 expression in single clones of MC38 cells. Setdb1 depleted cell clones (red color) were pooled together for in vivo experiments. (B) Western blot analysis to check Setdb1 expression in red color cell clone pools. C, CCK-8 assay checking the proliferation of MC38 cells transfected with lacZ or Setdb1 sgRNAs in culture.

FIG. 21. Setdb1 deficiency promotes the activation of infiltrated T-cells. (A) Representative flow cytometry analysis of CD69+ (T-cell activation marker) population of CD4+ T cells. (B) Scatter plot comparing the expression of CD69+ population of CD4+ T cells. (C) Representative flow cytometry analysis of CD62L+ (naive T-cell marker) population of CD4+ T cells. (D) Scatter plot comparing the expression of CD62L+ population of CD4+ T cells. (E) Representative flow cytometry analysis of CD44+ (T-cell activation marker) population of CD4+ T cells. (F) Scatter plot comparing the expression of CD44+ population of CD4+ T cells. (G) Representative flow cytometry analysis of CD69+ (T-cell activation marker) population of CD8+ T cells. (H) Scatter plot comparing the expression of CD69+ population of CD8+ T cells. (I) Representative flow cytometry analysis of CTLA4+ population of CD4+ T cells. (J) Scatter plots comparing the expression of CTLA4+ population of CD4+ T cells. (K) Representative flow cytometry analysis of CTLA4+ population of CD8+ T cells. (L) Scatter plots comparing the expression of CTLA4+ population of CD8+ T cells. (M) Representative flow cytometry analysis of T-bet+ population of CD4+ T cells. (N) Scatter plots comparing the expression of Tbet+ population of CD4+ T cells. (O) Representative flow cytometry analysis of T-bet+ population of CD8+ T cells. (P) Scatter plots comparing the expression of Tbet+ population of CD8+ T cells. For all flow cytometry experiments, the tumors from subcutaneous injection model were harvest and processed at day19 (sglacZ, n=6; sgAtf7ip, n=6). All data are mean±SEM. *, P≤0.05; **, P≤0.01.

FIG. 22. Atf7ip and Setdb1 expression didn't change upon interferon gamma stimulation. Western blot analysis to check Atf7ip and Setdb1 expression in MC38 cells after interferon gamma stimulation for 24 hours

FIG. 23. Setdb1 deficiency inhibited spliceosome activity and increased intron retention events. (A-B) Enrichment of genes associated with SPLICEOSOME (A) and SPLICEOSOMAL_SNRNP_ASSEMBLY (B) in MC38 cell with Setdb1 inhibition. (C) Number of transcripts with retained intron in MC38 cell with or without Atf7ip deficiency. The plot represents the MATS analysis using 6 biological replicates. Only events that passed the statistics threshold FDR<0.05 and PSI>0.1 (10% of the transcripts of a given gene) are taking into consideration. (D-E) Enrichment of genes associated with SPLICEOSOME (D) and SPLICEOSOMAL_SNRNP_ASSEMBLY (E) in human LUAD tumors with low SETDB1 expression.

FIG. 24. Systematic characterization of Atf7ip binding proteins in STRING database. (A) Atf7ip binding proteins network analysis identified in KP-IE2 cells. (B) Atf7ip binding proteins network analysis identified in MC38 cells.

FIG. 25. Atf7ip inhibition or Setdb1 inhibition synergies with anti-PD1 immunotherapy to inhibit tumor progression. All data are mean±SEM. **, p<0.01, ***, p<0.001.

FIG. 26. ERV sensor Rig-I inhibition partially blocks IFN upregulation upon knocking down Atf7ip or Setdb1. (A) RT-PCR analysis of Irf7 transcripts in MC38 or Rig-I deficient MC38 cell upon knocking down Atf7ip or Setdb1. (B) RT-PCR analysis of Irf9 transcripts in MC38 or Rig-I deficient MC38 cell upon knocking down Atf7ip or Setdb1. (C) RT-PCR analysis of Ifi44 transcripts in MC38 or Rig-I deficient MC38 cell upon knocking down Atf7ip or Setdb1. (D) RT-PCR analysis of Ifi204 transcripts in MC38 or Rig-I deficient MC38 cell upon knocking down Atf7ip or Setdb1. All data are mean±SEM. *, p<0.05, **, p<0.01, ***, p<0.001, ****, p<0.0001.

FIG. 27. ERV sensor Mda5 inhibition does not block IFN upregulation upon knocking down Atf7ip or Setdb1. (A) RT-PCR analysis of Irf7 transcripts in MC38 or Rig-I deficient MC38 cell upon knocking down Atf7ip or Setdb1. (B) RT-PCR analysis of Irf9 transcripts in MC38 or Rig-I deficient MC38 cell upon knocking down Atf7ip or Setdb1. (C) RT-PCR analysis of Ifi44 transcripts in MC38 or Rig-I deficient MC38 cell upon knocking down Atf7ip or Setdb1. (D) RT-PCR analysis of Ifi204 transcripts in MC38 or Rig-I deficient MC38 cell upon knocking down Atf7ip or Setdb1. All data are mean±SEM. *, p<0.05, **, p<0.01, ***, p<0.001, ****, p<0.0001.

FIG. 28. Anti-tumor immunity induced by Atf7ip inhibition depends on CD8 T cells but not NK cells. A-B, Tumor growth curves of MC38 tumors with or without Atf7ip deficiency in B6/J with or without CD8 T cells depletion. A-B, Tumor growth curves of MC38 tumors with or without Atf7ip deficiency in B6/J with or without NK cells depletion. All data are mean±SEM. **, p<0.01.

FIG. 29. Anti-tumor immunity induced by Setdb1 inhibition partially depends on CD8 T cells but not NK cells. A-B, Tumor growth curves of MC38 tumors with or without Setdb1 deficiency in B6/J with or without CD8 T cells depletion. A-B, Tumor growth curves of MC38 tumors with or without Setdb1 deficiency in B6/J with or without NK cells depletion. All data are mean±SEM. ***, p<0.001, ****, p<0.0001

### DETAILED DESCRIPTION OF THE DISCLOSURE

Unless defined otherwise herein, all technical and scientific terms used in this disclosure have the same meaning as commonly understood by one of ordinary skill in the art to which this disclosure pertains.

Every numerical range given throughout this specification includes its upper and lower values, as well as every narrower numerical range that falls within it, as if such narrower numerical ranges were all expressly written herein. As used herein, the singular forms “a” “and” and “the” include plural referents unless the context clearly dictates otherwise.

The disclosure includes all polynucleotide and amino acid sequences described herein. Each RNA sequence includes its DNA equivalent, and each DNA sequence includes its RNA equivalent. Complementary and anti-parallel polynucleotide sequences are included. Every nucleotide sequence encoding a polypeptide disclosed herein is encompassed by this disclosure. Amino acids of all protein sequences and all polynucleotide sequences encoding them are also included, including but not limited to sequences included by way of sequence alignments. Sequences from 80.00%-99.99% identical to any sequence (amino acids and nucleotide sequences) of this disclosure are included.

The present disclosure provides a method of inhibiting growth of cancer cells comprising inhibiting the expression or activity of ATF7IP and/or SETDB1. The inhibition of ATF7IP and/or SETDB1 may be carried out at any level, e.g., at the transcription/translation level, or the protein level. For example, specific small molecule inhibitors, including peptides, peptide-like molecules, and/or inhibitory RNAs may be used. Methods of inhibiting the activity also include targeted inhibition, such as site-directed mutagenesis or gene editing techniques (such as clustered regularly interspaced short palindromic repeats or CRISPR). In embodiments, a SETDB1 inhibitor my comprise a quinoline derivative, non-limiting examples of which are described in U.S. patent publication no. 20170354650 from which the entire description of SETDB1 inhibitors is incorporated herein by reference.

In an embodiment, this disclosure provides a method of enhancing anti-tumor immune response in an individual afflicted with a tumor comprising inhibiting the expression or activity of ATF7IP and/or SETDB1. The enhanced anti-tumor immune response may be mediated via restoration of immune surveillance in tumor cells. Data is provided herein to demonstrate there is significant upregulation in the expression of endogenous retroviral antigens (ERVs) upon Atf7ip and Setdb1 inhibition and subsequent anti-tumor immune response in immunocompetent hosts. For example, the expression of endogenous retroviral antigen gp70 and p15E may be increased. Further, upregulation of the expression of ERVs may activate type I interferon immune response.

In an embodiment, individuals subjected to inhibition of the expression or activity of ATF7IP and/or SETDB1 may exhibit reduced growth of cancer cells. Additionally, such individuals may become suitable for further therapy e.g., immune therapy. In an embodiment, this disclosure provides a method of inhibiting the growth of cancer cells comprising administering to an individual in need of treatment immune therapy in combination with inhibition of expression or activity of ATF7IP and/or SETDB1.

The term “cancer” as used herein refers to or describe the physiological condition in mammals in which a population of cells are characterized by unregulated cell growth. Examples of cancer include, but are not limited to, carcinoma, lymphoma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, melanoma, endometrial or uterine carcinoma, salivary gland carcinoma, kidney cancer, liver cancer, prostate cancer, vulval cancer, thyroid cancer, hepatic carcinoma and various types of head and neck cancer.

The term “therapeutically effective amount” as used herein refers to an amount of an agent sufficient to achieve, in a single or multiple doses, the intended purpose of treatment. For example, an effective amount to treat cancer is an amount sufficient to inhibit the growth of cancer cells. The exact amount desired or required will vary depending on the particular compound or composition used, its mode of administration and the like. An appropriate effective amount can be determined by one of ordinary skill in the art informed by the instant disclosure using only routine experimentation.

Within the meaning of the disclosure, “treatment” also includes relapse, or prophylaxis as well as the treatment of acute or chronic signs, symptoms and/or malfunctions. The treatment can be orientated symptomatically, for example, to suppress symptoms. It can be effected over a short period, be oriented over a medium term, or can be a long-term treatment, for example within the context of a maintenance therapy.

An agent for inhibiting or reducing the expression or activity of ATF7IP and/or SETDB1 may act by inhibiting nucleic acids or by inhibiting protein. For example, the agent may act at the gene level, mRNA level or at the protein level. Inhibition of ATF7IP and/or SETDB1 can comprise pharmacological inhibition of their activity. In certain approaches, pharmacologic inhibition comprises use of protein-specific small molecules for epigenetic regulation activity inhibition, or neutralizing antibodies, or other protein-specific biologics.

In an aspect, the disclosure includes inhibiting the expression or activity of ATF7IP and/or SETDB1 gene and/or inhibiting translation of RNA encoding these factors. Thus, in embodiments, the disclosure includes disruption of the ATF7IP and/or SETDB1 gene. Disruption of the gene may be performed using a chromosome editing approach, one non-limiting example of which comprises a CRISPR-based approach.

In various embodiments, a CRISPR-based method for genome editing is used to delete all or a portion of the ATF7IP and/or SETDB1 gene, or is used to insert one or more mutations into the gene, such that expression of ATF7IP and/or SETDB1 is reduced or preferably eliminated. Representative and non-limiting demonstrations of this approach are described below.

For example, any suitable CRISPR system can be used. In embodiments, a Type II CRISPR system is used. In embodiments, a Cas9 enzyme is used. In embodiments, the Cas9 is an S. pyogenes Cas9. Alternatives to Cas9 are known in the art and may be adapted for use in embodiments of this disclosure, such as Cas12a (formerly Cpf1), and may include enhanced CRISPR techniques, such as prime editing.

In various embodiments, the method of disclosure comprises introducing into the pertinent cells a CRISPR enzyme and a targeting RNA directed to the ATF7IP or SETDB1 gene, which may be a CRISPR RNA (crRNA) or a guide RNA, such as sgRNA. The sequence of the targeting RNA has a segment that is the same as or complementarity to any suitable CRISPR site in the ATF7IP or SETDB1 gene. In this regard, for Cas9 editing, the target sequence comprises a specific sequence on its 3′ end referred to as a protospacer adjacent motif or “PAM”. In an embodiment a CRISPR Type II system is used, and the target sequences therefore conform to the well-known N12-20NGG motif, wherein the NGG is the PAM sequence. Thus, in embodiments, a target RNA will comprise or consist of a segment that is from 12-20 nucleotides in length which is the same as or complementary to a DNA target sequence (a spacer) in the ATF7IP or SETDB1 gene. The 12-20 nucleotides directed to the spacer sequence will be present in the targeting RNA, regardless of whether the targeting RNA is a crRNA or a guide RNA. In embodiments, a separate trans-activating crRNA (tracrRNA) can be used to assist in maturation of a crRNA targeted to the ATF7IP or SETDB1 gene. Introduction of a CRISPR system into cells will result in binding of a targeting RNA/Cas9 (or other suitable enzyme) complex to the ATF7IP or SETDB1 gene target sequence so that the Cas9 can cut both strands of DNA causing a double strand break. The double stranded break can be repaired by non-homologous end joining DNA repair, or by a homology directed repair pathway, which will result in either insertions or deletions at the break site, or by using a repair template to introduce mutations, respectively. Double-stranded breaks can also be introduced into the ATF7IP or SETDB1 gene by expressing Transcription Activator-Like Effector Nucleases (TALENs) in the cells, Zinc-Finger Nucleases (ZFNs) in the pertinent cells.

In an embodiment, expression is inhibited by inhibiting transcription of RNA encoding ATF7IP and/or SETDB1. Transcription of mRNA may be inhibited by binding of a protein, such as an enzymatically inactive CRISPR enzyme, e.g., dCas9, to the DNA encoding the ATF7IP or SETDB1 mRNA, or DNA controlling its transcription.

In an embodiment, the disclosure includes interfering with the activity of mRNA encoding one or more of the ATF7IP and/or SETDB1, and/or inhibiting the transcription or translation of the mRNA, and as a result reducing expression of the enzyme(s). Reducing mRNA can involve introducing into cells that express the enzyme a molecule such as a polynucleotide that can inhibit translation of enzyme-encoding mRNA, and/or can participate in and/or facilitate RNAi-mediated reduction of the mRNA. For example, an antisense polynucleotide can be used to inhibit translation of the mRNA. Antisense nucleic acids can be DNA or RNA molecules that are complementary to at least a portion of the targeted mRNA. For example, the DNA or RNA molecules may be complementary to the portion of the mRNA that encodes for the ATF7IP and/or SETDB1. The DNA or RNA molecules may be from 5 to 15 nucleotides. The polynucleotides for use in targeting mRNA may be modified, such as, for example, to be resistant to nucleases.

This disclosure includes RNAi-mediated reduction in mRNA. RNAi-based inhibition can be achieved using any suitable RNA polynucleotide that is targeted to an enzyme-mRNA. For example, a single stranded or double stranded RNA, wherein at least one strand is complementary to the targeted mRNA, can be introduced into the cell to promote RNAi-based degradation of target mRNA. MicroRNA (miRNA) targeted to the mRNA can be used. A ribozyme that can specifically cleave target mRNA can be used. Small interfering RNA (siRNA) can be used. siRNA (or ribozymes) can be introduced directly, for example, as a double stranded siRNA complex, or by using a modified expression vector, such as a lentiviral vector, to produce an shRNA. As is known in the art, shRNAs adopt a typical hairpin secondary structure that contains a paired sense and antisense portion, and a short loop sequence between the paired sense and antisense portions. shRNA is delivered to the cytoplasm where it is processed by DICER into siRNAs. siRNA is recognized by RNA-induced silencing complex (RISC), and once incorporated into RISC, siRNAs facilitate cleavage and degradation of targeted mRNA. A shRNA polynucleotide used to suppress mRNA expression can comprise or consist of between 45-100 nucleotides, inclusive, and including all integers between 45 and 100, and all ranges there between. As an example, the portion of the shRNA that is complementary to the target mRNA can be from 21-29 nucleotides, inclusive, and including all integers between 21 and 29.

For delivering siRNA via shRNA, modified lentiviral vectors can be made and used according to standard techniques, given the benefit of the present disclosure. In certain approaches, modified lentiviruses are used to stably infect target cells, and may integrate into a chromosome in the targeted cells. For example, see Titus M A, Zeithaml B, Kantor B, Li X, Haack K, Moore D T, Wilson E M, Mohler J L, Kafri T. Dominant-negative androgen receptor inhibition of intracrine androgen-dependent growth of castration-recurrent prostate cancer. PLoS One 2012; 7(1):e30192.

In addition to lentiviral vectors, the described CRISPR and polynucleotide-based approaches, the disclosure includes use of one or more expression vectors, or by direct introduction of ribonucleoproteins (RNPs). Viral expression vectors may be used as naked polynucleotides, or may comprises any of viral particles, including but not limited to defective interfering particles or other replication defective viral constructs, and virus-like particles. In embodiments, the expression vector comprises a modified viral polynucleotide, such as from an adenovirus, a herpesvirus, or a retrovirus, such as an aforementioned lentiviral vector. In embodiments, any type of a recombinant adeno-associated virus (rAAV) vector may be used. In embodiments, a recombinant adeno-associated virus (rAAV) vector may be used. rAAV vectors are commercially available, such as from TAKARA BIO® and other commercial vendors, and may be adapted for use with the described systems, given the benefit of the present disclosure. In embodiments, for producing rAAV vectors, plasmid vectors may encode all or some of the well-known rep, cap and adeno-helper components. In certain embodiments, the expression vector is a self-complementary adeno-associated virus (scAAV). Suitable ssAAV vectors are commercially available, such as from CELL BIOLABS, INC.® and can be adapted for use in the presently provided embodiments when given the benefit of this disclosure. In embodiments, a transposon based system can be used. Any of the described approaches that use nucleases may also include a DNA repair template for use in, for example, homologous recombination with a target site.

In various embodiments, agents for inhibiting ATF7IP and/or SETDB1 (e.g., small molecule inhibitors, peptides and the like) may be used in the form of pharmaceutical compositions. The pharmaceutical compositions for parenteral administration include solutions, suspensions, emulsions, and solid injectable compositions that are dissolved or suspended in a solvent before use. The injections may be prepared by dissolving, suspending or emulsifying one or more of the active ingredients in a diluent. Examples of diluents are distilled water for injection, physiological saline, vegetable oil, alcohol, and a combination thereof. Further, the injections may contain stabilizers, solubilizers, suspending agents, emulsifiers, soothing agents, buffers, preservatives, etc. The injections, are sterilized in the final formulation step or prepared by sterile procedure. The pharmaceutical composition of the invention may also be formulated into a sterile solid preparation, for example, by freeze-drying, and may be used after sterilized or dissolved in sterile injectable water or other sterile diluent(s) immediately before use. The compositions described can include one or more standard pharmaceutically acceptable carriers. Some examples herein of pharmaceutically acceptable carriers can be found in: Remington: The Science and Practice of Pharmacy (2005) 21st Edition, Philadelphia, Pa. Lippincott Williams & Wilkins. The pharmaceutical composition of the invention may be administered by any route that is appropriate, including but not limited to parenteral or oral administration.

The method of the present disclosure may be carried out in an individual who has been diagnosed with cancer (“cancer patients”) or who is at a high risk of developing cancer. It may also be carried out in individuals who have a relapse or a high risk of relapse after being treated for cancer.

In an embodiment, this disclosure provides a method of inhibiting the growth of cancer cells comprising inhibiting the expression or activity of ATF7IP and/or SETDB1 at the transcription/translation/protein activity level.

In an embodiment, this disclosure provides a method of enhancing anti-tumor immune response in an individual comprising inhibiting the expression or activity of ATF7IP and/or SETDB1 at the transcription/translation/protein activity level.

In various embodiments, this disclosure provides a method of effecting one or more of the following: restoration of immune surveillance in tumor cells, upregulation in the expression of endogenous retroviral antigens (ERVs) (e.g., gp70 and p15E), upregulation of the expression of ERVs may activate type I interferon immune response comprising inhibiting the expression or activity of ATF7IP and/or SETDB1 at the transcription/translation/protein activity level.

In various embodiments, this disclosure provides a method of treating cancer comprising administering to an individual who has cancer, a therapeutically effective amount of an inhibitor of the expression or activity of ATF7IP and/or SETDB1, and optionally, further comprising administering immune therapy (e.g., administering a composition comprising one or more checkpoint inhibitors). Additionally, other anti-cancer therapies may also be carried out such as, chemotherapy, radiation, surgery and the like.

In various embodiments, this disclosure provides a method of inhibition of growth of cancer cells or enhancing anti-tumor immune response comprising contacting the cells with an effective amount of an inhibitor of the expression or activity of ATF7IP and/or SETDB1.

In an aspect, this disclosure provides a method of identifying patients who are suited for immune checkpoint inhibition (ICI) therapy. The method comprises screening individuals who are diagnosed with cancer for the expression/activity of ATF7IP or SETDB1 and if the levels of expression or activity of these factors is found to be low (compared to a reference), then considering the ICI therapy to be suitable for the individual, and if the level of expression or activity of these factors is found to be high then identifying the individuals to not be suitable for ICI therapy. In an embodiment, the patients who have high level of expression or activity of ATF7IP and/or SETDB1 can be subjected to lowering the expression or activity of these factors prior to initiation of ICI. This can be followed by administration of immune therapy. Alternatively, instead of screening for the expression or activity of ATF7IP or SETDB1, a factor downstream of their activation can be screened.

Various immune therapies are known in the art. For example, cytotoxic T-lymphocyte-associated protein 4 (CTLA-4) or programmed death 1 (PD-1) pathway may be targeted as immune checkpoint inhibitors. In particular, the programmed death receptor 1 (PD-1) is a T-cell surface receptor that is expressed on T cells, B cells, natural killer cells (NK), activated monocytes and dendritic cells. The role of PD-1 in normal human physiology is to limit autoimmunity by acting as a co-inhibitory immune checkpoint expressed on the surface of T cells and other immune cells, including tumor-infiltrating lymphocytes. It has two ligands: programmed death receptor ligand 1 (PD-L1/B7-H1) and 2 (PD-L2/B7-DC). Examples of T cell-based immunotherapies include adoptive cell transfer therapies in which patients are infused with their own immune cells (e.g., T cells include enriched populations of tumor-reactive T cells, genetically-engineered CAR-T cells (chimeric antigen receptor T cells) or T cell receptor-engineered T cells, and natural killer cells (NK cells; FATE-NK100)); cancer vaccines including dendritic cell (DC)-based vaccines; or antibody therapies directed against immune checkpoints PD-1 (e.g., nivolumab, pembrolizumab, cemiplimab, pidilizumab, PDR001, MEDI4736/duralumab, ABBVI-181), PD-L1 (e.g., atezolizumab, durvalumab, avelumab), CTLA-4 (e.g., ipilimumab, tremelimumab), LAG-3 (e.g., TSR-033), Tim-1 (e.g., TSR-022), or immune-activating antibodies (e.g., directed against 41BB (e.g., utomilumab); Ox40 (e.g., PF-04518600, ABBV-368); and CD122 (e.g., NKTR-262, NKTR-214).

The present methods may be used alone, or with other modalities, including chemotherapeutic agents, surgery, radiation and the like. Therefore, an inhibitor that blocks ATF7IP and/or SETDB1 may be used in combination with existing therapies to provide a new treatment strategy to block the key steps of cancer immune evasion.

The level of expression of ATF7IP and/or SETDB1 in a sample obtain from an individual can be determined using routine techniques. In embodiments, the level of expression of ATF7IP and/or SETDB1 in a sample obtain from an individual can be compared to a control. Any suitable control, such as a control value, can be used. In embodiments, the control value comprises the level of expression of ATF7IP and/or SETDB1 in a sample obtained from an individual who does not have cancer, and/or from a sample of tissue or other biological material that is not cancerous. In an embodiment the disclosure therefore provides a method of identifying if an individual is suited for immune therapy. This approach comprises determining the expression or activity of ATF7IP and/or SETDB1 in a tumor sample obtained from the individual, and if the level is the same or lower that the level from a control sample, the method further comprises identifying the individual to be suitable for immune therapy. If the expression level is higher than the level from the control sample, the method further comprises identifying the individual to be not suitable for immune therapy. For this individuals identified as being suitable for immune therapy, the method may further comprise administering to the individual at least one immune therapy, as further described herein.

The following examples further describe the disclosure. These examples are intended to be illustrative and not limiting in any way.

### EXAMPLE 1

This example describes materials and methods used in an epigenetic CRISPR loss of function screen in an antigen silenced immune escaped lung adenocarcinoma tumor model. Using the materials and methods, we identified activating transcription factor 7 interacting protein (Atf7ip) and its partner histone H3-K9 methyltransferase 4 (Setdb1) as therapeutic targets to augment tumor immunogenicity. Functional and mechanistic studies demonstrated that Atf7ip-Setdb1 deficiency stimulated anti-tumor immunity in multiple tumor models. Thus, the disclosure provides a method for using ATF7IP and SETDB1 as immunotherapeutic targets in cancer patients.

Cell culture, plasmid construction, and lentivirus infection. HEK-293T cells and MC38 cells were cultured in Dulbecco's Modified Eagle Medium (DMEM, Gibco) with 10% fetal bovine serum (FBS). YUMM.17 was cultured as previously described (Meeth et al., 2016). Mouse lung ADC lines KP, KP-IE2 and KP-LucOS (C57BL/6 background) were cultured in Roswell Park Memorial Institute (RPMI) 1640 (Gibco) with 10% FBS. All cell lines were tested as mycoplasma negative from Universal Mycoplasma Detection Kit (ATCC® 30-1012K™). Plasmids pLenti-Cas9-Puro, pXPR-GFP-Blast, lentiCRISPRv2 neo, PSPAX2 and PMD2.G were purchased from Addgene.

The sgRNAs of mouse Atf7ip and Setdb1 were cloned into pXPR-GFP-Blast vector using Gibson Assembly kit (E2611L, NEB) or cloned into lentiCRISPRv2 neo vector using T4 DNA ligase (M0202S, NEB). The sgRNA sequences are as shown below in Table 1, provided as DNA sequences. The disclosure includes each sequence in its RNA form, wherein each T is replaced with a U.

TABLE 1
sgRNA Sequences

| sgRNA | Sequence | SEQ ID NO |
|---|---|---|
| sgAtf7ip-1 | 5′-ATTCTTCATCCATATATCGC-3′ | 1 |
| sgAtf7ip-2 | 5′-GGCAGTGCTCACCGAGCTGC-3′ | 2 |
| sgAtf7ip-3 | 5′-AAGATCACAAGTTATACTGG-3′ | 3 |
| sgSetdb1-1 | 5′-TGCCTATCCAAACCGCCCAA-3′ | 4 |
| sgSetdb1-2 | 5′-CAGAACTCCAAAAGACCAGA-3′ | 5 |
| sgSetdb1-3 | 5′-AGGACTAAGACATGGCACAA-3′ | 6 |

To generate lentivirus, HEK-293T cells were co-transfected with pLenti-Cas9-Puro, pXPR-GFP-sgRNA-Blast and packaging plasmids PSPAX2 and PMD2.G using Lipofectamine 3000 (Invitrogen). Viral particles with the cell culture supernatant were filtered with 0.45-μm filters (Corning) to remove cellular debris. KP, KP-SQ, KP-LucOS and MC38 cells were infected with viral supernatants in the presence of 10 ug/ml polybrene. Stable cell lines were selected and maintained in cell culture media containing 5 μg/mL puromycin, 5 ug/mL blasticidin or 600 ug/mL G418.

Epigenetic CRISPR screen using an immune escaped KP-IE2 lung cancer cell line. KP-IE2 lung ADC cells with Cas9 activity were infected at a MOI of 0.2 with lentivirus generated from the epigenetic libraries for at least 1000-fold coverage (1000 cells per sgRNA construct) in each infection replicate. Transduced KP-IE2 cells were expanded in vitro for 2 weeks, and then both 15% of SIINFEKL (SEQ ID NO:47) high expression population (SIINFEKL (SEQ ID NO:47)_high) and 15% low expression population (SIINFEKL (SEQ ID NO:47)_low) were sorted out. Genomic DNAs of these two populations were extracted using the DNeasy Blood & Tissue kit (69506, Qiagen). sgRNA cassettes were amplified by PCR, and NGS sequencing was performed on an Illumina HiSeq to determine sgRNA abundance.

Data analysis for CRISPR screen. Cutadapt (v1.18) was applied to trim adaptor sequences, and untrimmed reads were discarded. Then the sequences after the 20-base sgRNAs were cut using fastx-toolkit (v0.0.13) (http://hannonlab.cshl.edu/fastx_toolkit/index.html), sgRNAs were mapped to the annotation file (0 mismatch), and read count tables were made. The count tables were normalized based on their library size factors using DESeq2 (Love et al., 2014), and differential expression analysis was performed. Furthermore, MAGeCK (0.5.8) (Li et al., 2014) was applied to normalize the read count tables based on median normalization and fold changes, and significance of changes in the conditions was calculated for genes and sgRNAs. ClusterProfiler R package (v3.6.0) were applied to perform pathway analysis and Gene Set Enrichment Analysis (Yu et al., 2012). R (v3.1.1) was used to perform all downstream statistical analyses and generate plots (http://www.r-project.org).

Animal studies. All mice work was reviewed and approved by the Institutional Animal Care and Use Committee (IACUC) at NYU School of Medicine. All mice were housed and cared in specific-pathogen-free facilities. Six-week old B6/J WT mice were purchased from Jackson Laboratories. Six-week old NU/NU Nude mice were purchased from Charles River Laboratories. For MC38 in vivo model, 1 million cells were resuspended in phosphate buffered saline (PBS) and subcutaneously inoculated into the flanks of B6/J and NU/NU Nude mice. Tumor size was measured every 3 days using calipers to collect maximal tumor length and width. Tumor volume was calculated with the following formula: (L×W2)/2. For KP-IE2 and KP-LucOS in vivo model, 1 million cells were injected into each mouse via tail vein. Tumor formation and progression were monitored by Magnetic resonance imaging (MM). CO2 inhalation was used to euthanize mice when the tumor samples were harvested.

MRI quantification. Mice were anesthetized with isoflurane, and then lung MRI was performed using BioSpec USR70/30 horizontal bore system (Bruker) to scan 24 consecutive sections. Tumor volume in the whole lung was quantified using 3-D slicer software to reconstruct MM volumetric measurements, as described previously (Chen et al., 2012). Acquisition of the MM signal was adapted based on cardiac and respiratory cycles to minimize the mice motion effects during the imaging process.

RNA-seq and data analyses. RNA-seq of MC38 cells with or without Atf7ip deficiency and RNA-seq of MC38 cells with or without Setdb1 deficiency were performed in NYU School of Medicine Genome Technology Core. STAR 2.4.2a (Dobin et al., 2013) was applied to align the RNA-seq samples to the reference mouse genome (mm9) and count the number of reads that map to each gene in the ensembl GRCm38.80 gene model. R (v.3.5.1) (http://www.R-project.org/) and the DESeq2 package (v.1.10.0) were used to perform differential gene expression analysis among different sample groups (Anders and Huber, 2010). Gene set enrichment analysis was done using GSEA (v.3.0) and gene sets from MSigDB (v.5.0). We used the ‘preranked’ algorithm to analyze gene lists ranked by the negative decadic logarithm of P values multiplied by the value of log2FC obtained from the differential-expression analysis with DESeq2.

TCGA RNA-seq data analysis. Level 3 RNA-seq data of TCGA was obtained through the TCGA portal. Data was sorted based on the expression level of ATF7IP or SETDB1, and the samples were separated into quarters. The top 25% expression group (high expression) was compared with the low 25% expression group (low expression) by GSEA analysis as outlined in the RNA-seq data analysis section. The gene list for GSEA input was ranked by the value of log2FC, where FC was defined by the ratio of low expression group to high expression group.

ChIP-seq and ATAC-seq. Chromatin immunoprecipitation (ChIP) was performed in MC38 cells using ChIP-IT High Sensitivity Kit (53040, Active Motif) following the manufacturer's instructions. Antibodies against Atf7ip, Setdb1 and Histone H3 (tri methyl K9) (A300-169A, Bethyl Laboratories; 11231-1-AP, Proteintech; ab8898, Abcam) were used. ChIP DNA was purified and sent to NYU School of Medicine Genome Technology Center for library construction and sequencing. For ATAC-seq, freshly harvested cells were directly sent to NYU School of Medicine Genome Technology Center for library construction and sequencing.

ChIP-Seq and ATAC-Seq data analysis. Bowtie2 (v2.2.4) (Langmead and Salzberg, 2012) was applied to map all the reads from sequencing to the reference genome and Picard tools (v.1.126) (broadinstitute.github.io/picard/) was used to remove duplicate reads. Low-quality mapped reads (MQ<20) were discarded from the analysis. BEDTools (v.2.17.0) (Quinlan and Hall, 2010) and the bedGraphToBigWig tool (v.4) were applied to generate read per million (RPM) normalized BigWig files. MACS (v1.4.2) (Zhang et al., 2008) was used to perform peak calling and BEDTools was applied to creat peak count tables. DESeq2 (Love et al., 2014) was applied to perform differential peak analysis. ChIPseeker (v1.8.0) (Yu et al., 2015) R package was used for peak annotations and motif discovery was performed with HOMER (v4.10) (Heinz et al., 2010). ngs.plot (v2.47) (Heinz et al., 2010). ChIPseeker were applied for TSS site visualizations and quality controls. clusterProfiler R package (v3.0.0) (Yu et al., 2012) was used to perform KEGG pathway analysis and Gene Ontology (GO) analysis. To compare the level of similarity among the samples and their replicates, two methods were used: principal-component analysis and Euclidean distance-based sample clustering. Downstream statistical analysis and generating plots were performed in R environment (v3.1.1) (https://www.r-project.org/).

Tumor-infiltrating immune cells profiling. Mice were euthanized. Tumors were minced and digested in Hank's Balanced Salt Solution with collagenase D (11088866001, Roche) and DNase I (10104159001, Roche) at 37° C. for 30 minutes. After digestion, the whole tumors were filtered through 70-μm cell strainers (Thermo Fisher Scientific) to obtain single-cell suspensions. Suspended cells were treated with 1× RBC lysis buffer (BioLegend) to lyse red blood cells. Live cells were stained with a LIVE/DEAD Fixable Aqua Dead Cell Stain kit (Molecular Probes). Well processed cell pellets were resuspended in PBS with 2% FBS for FACS analysis. Cells were stained with cell surface markers and then were fixed/permeabilized with Fixation/Permeabilization kit (eBioscience). Well stained cells were collected by BD Biosciences LSRFortessa and the data was analyzed with FlowJo software. The gating strategy was described previously (Misharin et al., 2013).

Flow antibodies. Tumor-infiltrating immune cells were stained with fluorochrome-coupled antibodies against mouse CD45 (clone 30-F11, BioLegend), CD3 (clone 17A2, BioLegend), CD4 (clone GK1.5, BioLegend), CD8 (clone 53-6.7, BioLegend), CD44 (clone IM7, BioLegend), CD62L (clone MEL-14, Biolegend), CD69 (clone H1.2F3, BioLegend), T-bet (clone 4B10, BioLegend), CD279 (PD-1) (clone 29F.1A12, BioLegend), CD152 (CTLA-4) (clone UC10-4B9, eBioscience).

Western blots and antibodies. Cells were lysed in RIPA buffer (Pierce) with protease/phosphatase inhibitor cocktail (Thermo Fisher Scientific). Protein concentration was determined by BCA assay (Pierce). Equivalent proteins from each sample were loaded in 4%-12% Bis-Tris gels (Invitrogen), transferred to nitrocellulose membranes, and immunoblotted with primary antibodies against Cas9 (MA1-202, Thermo Fisher Scientific), Atf7ip (A300-169A, Bethyl Laboratories), Setdb1 (11231-1-AP, Proteintech), Lamin B1 (sc-374015, Santa Cruz Biotechnology), Stat2 (4597s, CST), Rig-I (3743s, CST), Mda5 (5321s, CST), Phopho-Irf7 (24129s, CST) , Irf7 (72073s, CST) , Irf9 (28845s, CST) and β-actin (Ab8227, Abcam). IRDye 800-labeled goat anti-rabbit IgG and IRDye 680-labeled goat anti-mouse IgG (LI-COR Biosciences) were applied as secondary antibodies. The membranes were imaged with an Odyssey platform (LI-COR Biosciences).

MS identification of Atf7ip interactions. KP-IE2 and MC38 cells were lysed with IP lysis buffer (Pierce). Cell lysates were incubated with Rabbit IgG antibody (2729s, CST), anti-Atf7ip antibody (A300-169A, Bethyl Laboratories) and A-agarose beads (Pierce, 20333) overnight. The agarose resin with immunoprecipitated proteins was washed with IP lysis buffer 3 times. Samples were then washed 3 times with 100 uM Ammonium Bicarbonate. Samples were reduced with DTT at 57° C. for 1 hour (2 μl of 0.2 M). Samples were alkylated with Iodoacetamide at RT in the dark for 45 minutes (2 μl of 0.5 M). 200 ng of sequencing grade modified trypsin (Promega) was added to each gel sample. Digestion proceeded overnight on a shaker at RT. Beads were removed and Peptides extracted. A slurry of R2 20 μm Poros beads (Life Technologies Corporation) in 5% formic acid and 0.2% trifluoroacetic acid (TFA) was added to each sample at a volume equal to that of the ammonium bicarbonate added for digestion. The samples shook at 4° C. for 3 hours. The beads were loaded onto equilibrated C18 ziptips (Millipore) using a microcentrifuge for 30 seconds at 6000 rpm. Gel pieces were rinsed three times with 0.1% TFA and each rinse was added to its corresponding ziptip followed by microcentrifugation. The extracted poros beads were further washed with 0.5% acetic acid. Peptides were eluted by the addition of 40% acetonitrile in 0.5% acetic acid followed by the addition of 80% acetonitrile in 0.5% acetic acid. The organic solvent was removed using a SpeedVac concentrator and the sample reconstituted in 0.5% acetic acid. 1/30th of each sample was analyzed individually. LC separation online with MS using the autosampler of an EASY-nLC 1000 (Thermo Scientific). Peptides were gradient eluted from the column directly to a Lumos Mass spectrometer using a 95 min gradient (Thermo Scientific). High resolution full MS spectra were acquired with a resolution of 240,000, an AGC target of 1e6, with a maximum ion time of 50 ms, and scan range of 400 to 1500 m/z. All MS/MS spectra were collected using the following instrument parameters: Ion trap scan rate of Rapid, AGC target of 6e4, maximum ion time of 18 ms, one microscan, 2 m/z isolation window, fixed first mass of 110 m/z, and NCE of 30. MS/MS spectra were searched using a Uniprot Human database plus IgG and supplied sequences using Sequest within Proteome Discoverer.

Quantitative RT-PCR. Total RNA was extracted with RNeasy Plus Mini Kit (Qiagen), and cDNA was constructed with a High-Capacity cDNA Reverse Transcription Kit (Applied Biosystems). Quantitative PCR was run in Real-Time PCR System (Applied Biosystems), and transcripts were normalized with internal control actin. All the samples were run in triplicates. The RT-PCR primer sequences are as shown in Table 2.

TABLE 2
RT-PCR Primer Sequences

| RT-PCR | | SEQ |
|---|---|---|
| Primer | Sequence | ID NO |
| p15E-F | 5′-CCACACTGGCGTAGTAAGGG-3′ | 7 |
| p15E-R | 5′-GTTGAGAATGCAGGGTCCGA-3′ | 8 |
| Gp70-F | 5′-TGACCTTGTCCGAAGTGACC-3′ | 9 |
| Gp70-R | 5′-TAGGACCCATCGCTTGTCTT-3′ | 10 |
| SQ-F | 5′-ACGGCCAATTCATTCACTTC-3′ | 11 |
| SQ-R | 5′-ACTCTTTGGTGGTGGACTGG-3′ | 12 |
| LucOS-F | 5′-ATCCATCTTGCTCCAACACC-3′ | 13 |
| LucOS-R | 5′-TCGCGGTTGTTACTTGACTG-3′ | 14 |
| mIrf7-F | 5′-CCTCTGCTTTCTAGTGATGCCG-3′ | 15 |
| mIrf7-R | 5′-CGTAAACACGGTCTTGCTCCTG-3′ | 16 |
| mIrf9-F | 5′-CAACATAGGCGGTGGTGGCAAT-3′ | 17 |
| mIrf9-R | 5′-GTTGATGCTCCAGGAACACTGG-3′ | 18 |
| mTnf-F | 5′-GGTGCCTATGTCTCAGCCTCTT-3′ | 19 |
| mTnf-R | 5′-GCCATAGAACTGATGAGAGGGAG-3′ | 20 |
| mOas1a-F | 5′-GAGGTGGAGTTTGATGTGCTGC-3′ | 21 |
| mOas1a-R | 5′-GTGAAGCAGGTAGAGAACTCGC-3′ | 22 |
| mOas1b-F | 5′-CTGTGCTGACCTCAGAGAAGTC-3′ | 23 |
| mOas1b-R | 5′-TGCCCTTGAGTGTGGTGCCTTT-3′ | 24 |
| mOas1c-F | 5′-GACTTCCGACATCAAGAGGTCTG-3′ | 25 |
| mOas1c-R | 5′-ATCCAGGTCTGAGCCTCCTTTG-3′ | 26 |
| mOas1g-F | 5′-GAGTCTCATCCGCCTGGTCAAA-3′ | 27 |
| mOas1g-R | 5′-CCAGGCATAGACAGTGAGTAGC-3′ | 28 |
| mOas3-F | 5′-TTCTCTGCCAGCTTCGGAAAGC-3′ | 29 |
| mOas3-R | 5′-CTCTGAAGGCAGACTTGTGACC-3′ | 30 |
| mIfi44-F | 5′-ATGCACTCTTCTGAGCTGGTGG-3′ | 31 |
| mIfi44-R | 5′-TCAGATCCAGGCTATCCACGTG-3′ | 32 |
| mIfi2712a-F | 5′-CTTCACTGGGACAGGCATTGCA-3′ | 33 |
| mIfi2712a-R | 5′-CCTGCTGATTGGAGTGTGGCTA-3′ | 34 |
| mIfi47-F | 5′-GCAGAGAATGCTATCCTGGAGG-3′ | 35 |
| mIfi47-R | 5′-CAGCGGATTCATCTGCTTCGTG-3′ | 36 |
| mIfi203-F | 5′-CCAATGTCAGGTGTGAACCAGG-3′ | 37 |
| mIfi203-R | 5′-AGTCTGGGTTGAGTGGCTTTCC-3′ | 38 |
| mIfi204-F | 5′-TGCAAATGCCAGCCCTAAGA-3′ | 39 |
| mIfi204-R | 5′-CCATTTTCCACTCCCCACCA-3′ | 40 |
| mI17-F | 5′-CAGGAACTGATAGTAATTGCCCG-3′ | 41 |
| mI17-R | 5′-CTTCAACTTGCGAGCAGCACGA-3′ | 42 |
| mActin-F | 5′-CTGTCCCTGTATGCCTCTG-3′ | 43 |
| mActin-R | 5′-ATGTCACGCACGATTTCC-3′ | 44 |
| hACTIN-F | 5′-CACCATTGGCAATGAGCGGTTC-3′ | 45 |
| hACTIN-R | 5′-AGGTCTTTGCGGATGTCCACGT-3′ | 46 |

Statistical analysis. GraphPad Prism 7 was used for all statistical analyses. Data was analyzed by Student's t-test (two tailed). Survival analysis was performed by Kaplan-Meier method and log rank (Mantel-Cox) test. P<0.05 was considered significant. Error bars represent standard error of the mean (SEM).

Data access. NGS data for CRISPR screen, RNA-seq data, ChIP-seq data and ATAC-seq data have been deposited in the National Center for Biotechnology Information's Gene Expression Omnibus and are accessible through GEO Series accession number GSE127205 (ncbi.nlm.nih.gov/geo/query/acc.cgi?acc=GSE127205), GSE127232 (ncbi.nlm.nih.gov/geo/query/acc.cgi?acc=GSE127232), GSE133604 (ncbi.nlm.nih.gov/geo/query/acc.cgi?acc=GSE133604), and GSE138571 (ncbi.nlm.nih.gov/geo/query/acc.cgi?acc=GSE138571).

### EXAMPLE 2

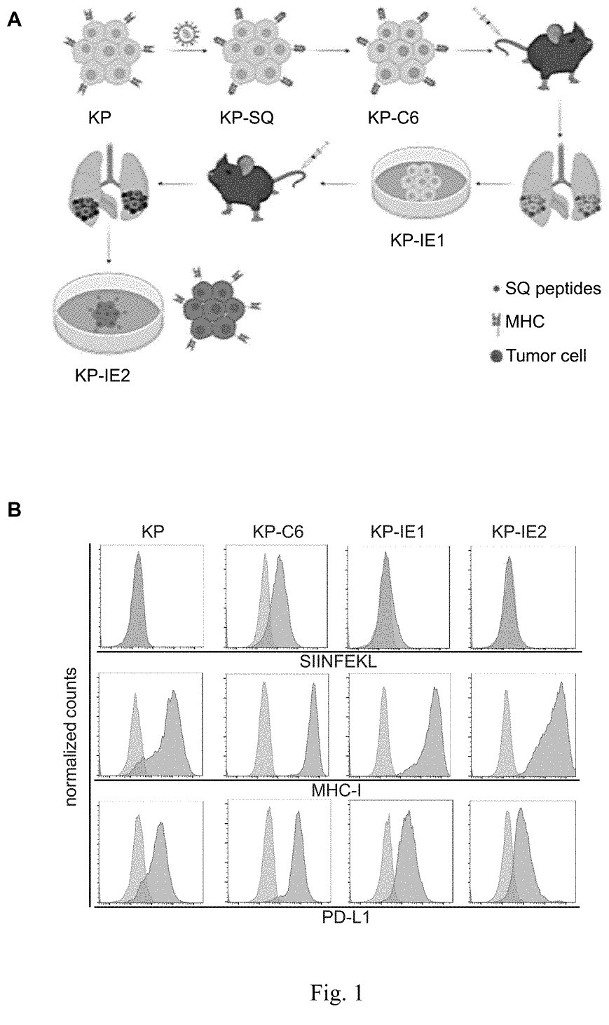

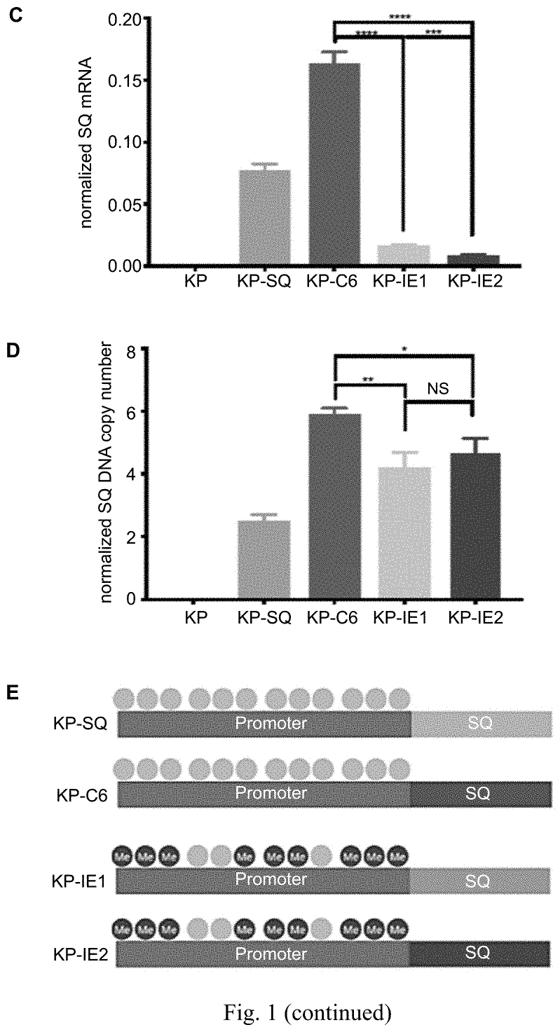

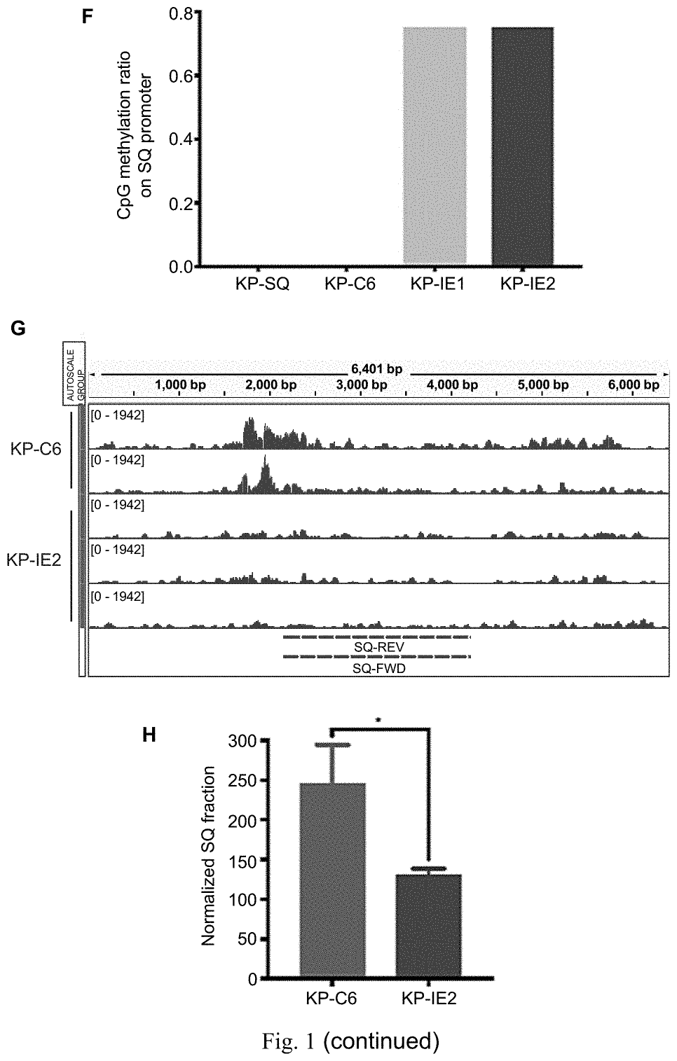

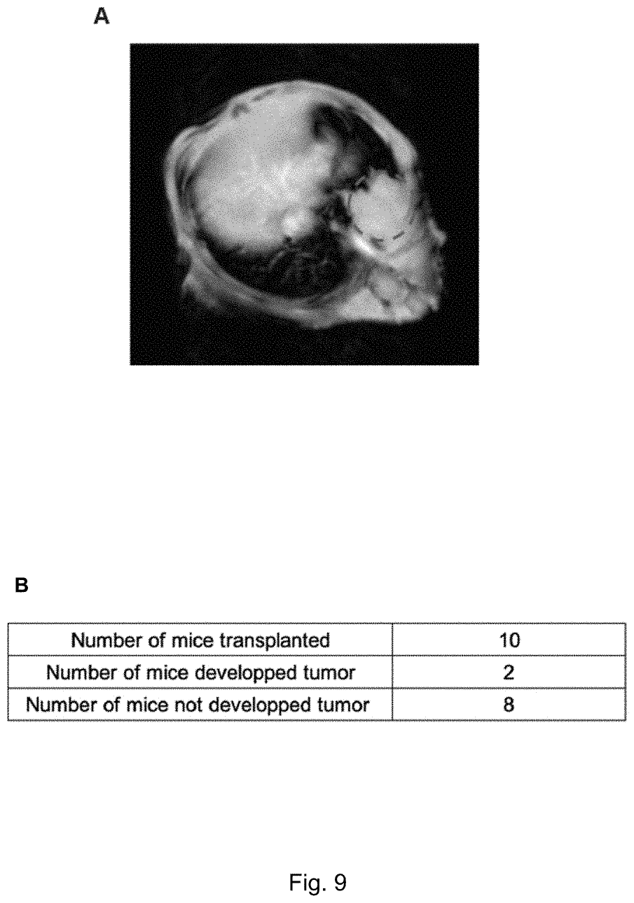

Silencing of tumor antigen expression contributes to tumor immune evasion. Previously we established KrasG12D/Tp53−/− (KP) murine lung cancer cell line in C57BL6/J (B6/J) background (Li et al., 2020). Allografted tumors established with KP cell line are very malignant and grow rapidly in B6/J immunocompetent mice, indicating the loss of immunogenicity of tumor cells. To mimic immunoediting in driving the outgrowth of immune evasive tumor cells that initially were highly immunogenic, KP cells were engineered to express exogenous antigen SQ by lentiviral infection (FIG. 1A). SQ coding sequence consists of ovalbumin-SIINFEKL (SEQ ID NO:47) (SIN, OVA₂₅₇₋₂₆₄), which enables the monitoring of SQ expression and presentation in tumor cells. The single cell clone (KP-C6) expressing high level of SIN was injected to B6/J immunocompetent mice to undergo tumor immunoediting. Injected KP-C6 cells formed tumors in the lungs of some grafted mice (2/10) after 10 months as indicated by MRI imaging (FIG. 1A; FIG. 9A, B). The tumor nodule was harvested to establish the cell line KP-IE1 (1st generation of immune escaped model) (FIG. 1A). KP-IE1 cells formed tumors only 3 weeks after injection into B6/J mice, from which the tumor nodule was collected again to establish the cell line KP-IE2 (2nd generation of immune escaped model) (FIG. 1A).

We next evaluated the characteristics of our immune escaped tumor model. While we observed no difference in cell membrane MHC-I expression, we found a significant inhibition of SIN presentation in both KP-IE1 and KP-IE2 cells (FIG. 1B). Notably, we found no change in PD-L1 expression (FIG. 1B). In addition, SQ was transcriptionally inhibited while its genomic copy number showed no dramatic change (FIG. 1C, D). SQ is under the control of CMV promoter in the lentiviral vector, and CpG methylation analysis indicated CMV promoter was hypermethylated in the KP-IE1 and KP-IE2 cell lines (FIG. 1E, F). Furthermore, the SQ region in the chromatin was less accessible in these cell lines (FIG. 1G, H), suggesting more heterochromatin formed in the SQ genomic region during tumor immune escape. These results support that KP-IE2 escaped immune surveillance through silencing antigen expression.

### EXAMPLE 3

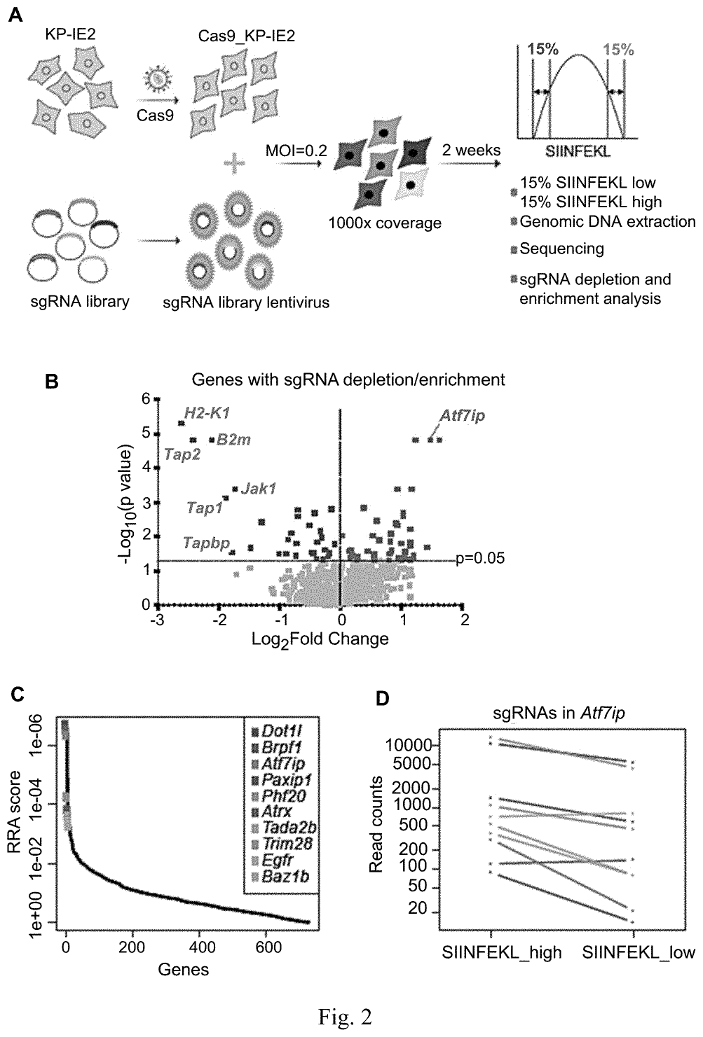

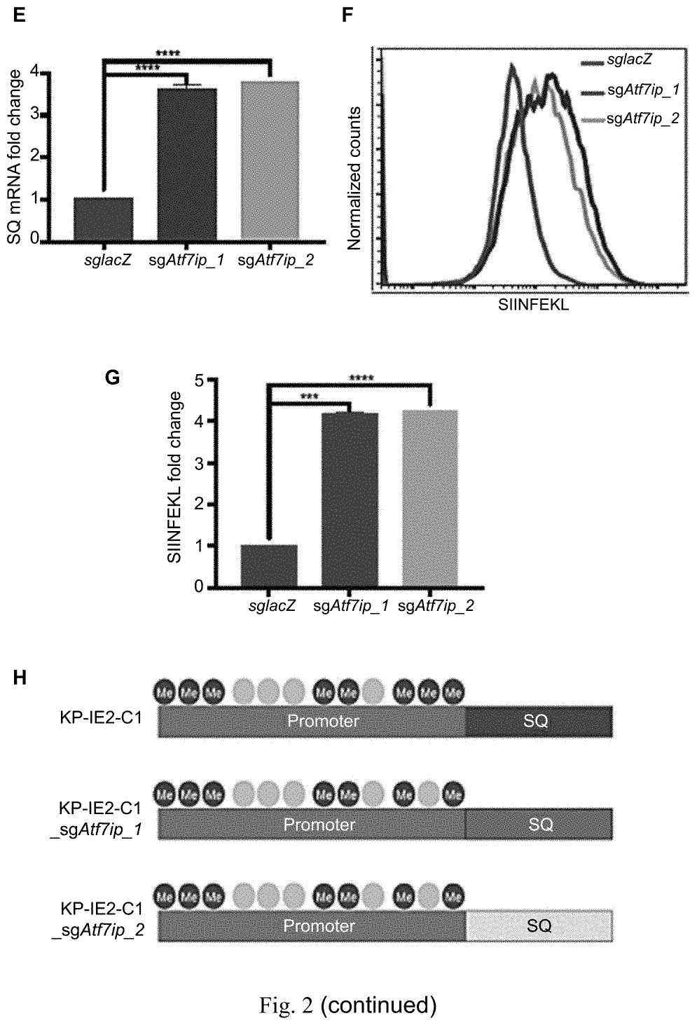

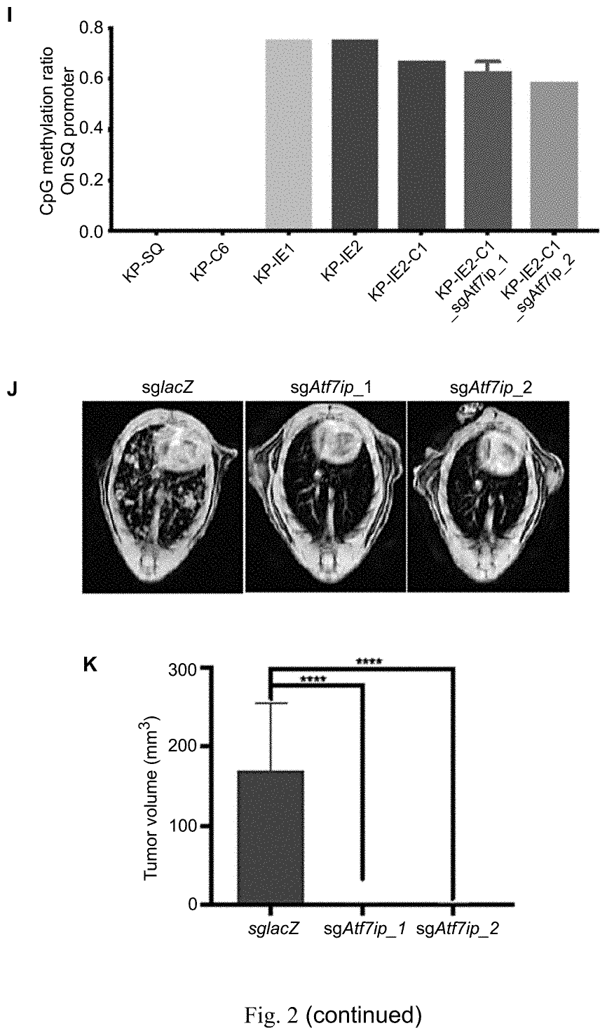

CRISPR screen identifies Atf7ip as an epigenetic factor controlling tumor antigen expression and presentation. Epigenetic modulators have been shown to regulate tumor cell antigen expression and presentation (Chiappinelli et al., 2015; Wang et al., 2020). To identify novel epigenetic modifiers which are critical for tumor antigen expression and presentation, we performed epigenome-wide CRISPR loss-of-function screen in immune escaped KP-IE2 cells (FIG. 2A). The sgRNA library contains sgRNAs targeting 524 epigenetic genes, described previously (Li et al., 2020). KP-IE2 cells were infected with the lentivirus pool and cultured for 2 weeks, and sorted based on 15% of SIINFEKL (SEQ ID NO:47) high expression population (SIINFEKL (SEQ ID NO:47)^{high}) and 15% low expression population (SIINFEKL (SEQ ID NO:47)^{low}) (FIG. 2A). Through comparing sgRNAs extracted from SIINFEKL (SEQ ID NO:47)^{high} population with SIINFEKL (SEQ ID NO:47)^{low} population, we identified the epigenetic targets whose loss-of-function can promote or inhibit SIINFEKL (SEQ ID NO:47) expression and presentation (FIG. 2A). For example, sgRNAs targeting H2-K1, Tap1, Tap2, B2m and Tapbp were enriched in SIINFEKL (SEQ ID NO:47)^{low} population (FIG. 2B), consistent with their established role in positively regulating antigen presentation. These findings validated our CRISPR screen model. On the other side, sgRNAs targeting Atf7ip was enriched in SIINFEKL (SEQ ID NO:47)^{high} population (FIG. 2B-D), indicating a role in negatively regulating antigen expression and presentation. Indeed, both SQ expression and SIINFEKL (SEQ ID NO:47) presentation were significantly increased in KP-IE2 cells upon Atf7ip inhibition (FIG. 2E-G). Moreover, CpG methylation analysis indicates the CMV promoter was still hypermethylated upon Atf7ip inhibition (FIG. 2H, I), suggesting that Atf7ip deficiency functions downstream of the DNA methylation to reverse the tumor antigen silencing.

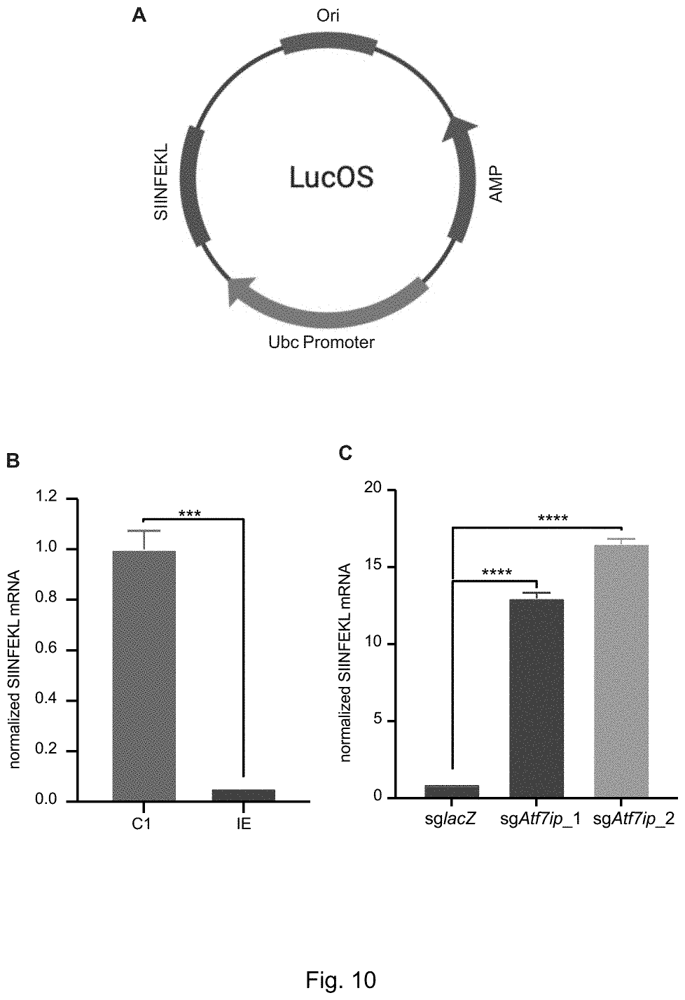

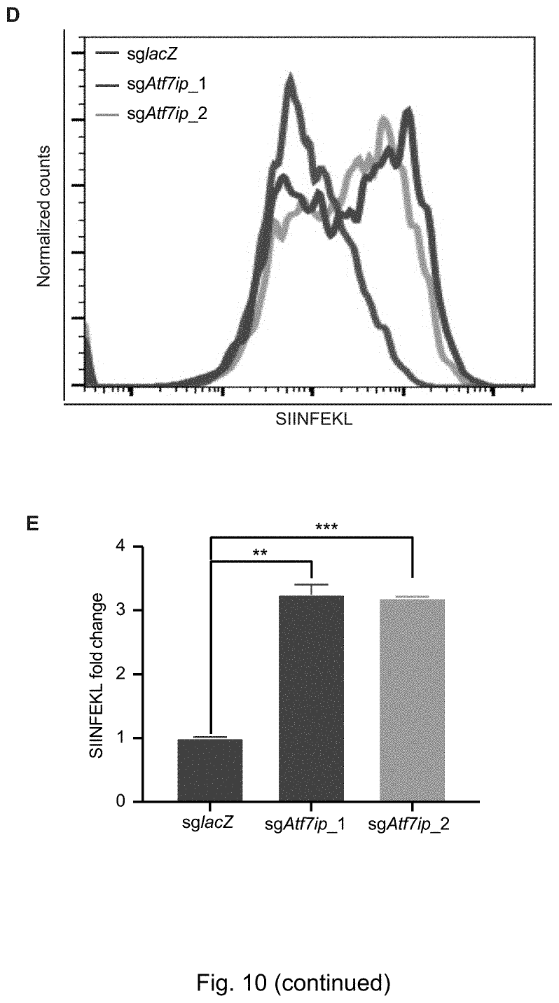

To exclude the possibility that the regulatory effect of Atf7ip is CMV promoter specific, we applied an alternative model. LucOS is a lentiviral vector expressing the T cell antigen SIYRYYGL (SIY) and two antigens from ovalbumin-SIINFEKL (SEQ ID NO:47) (SIN, OVA257-264) and OVA323-339 (DuPage et al., 2011) under the control of UbC promoter (FIG. 10A). In order to investigate whether Atf7ip inhibition could also restore tumor antigen expression in immune escaped KP-LucOS model, we established immune escaped KP-LucOS cell line with silenced antigen expression (FIG. 10B). We infected KP lung adenocarcinoma cells with LucOS lentivirus and orthotopically injected KP-LucOS cells into B6/J mice. The lung tumor nodules developed in the mice were harvested for establishment of the immune escaped cell line. Indeed, consistent with previous work, the immune escaped tumor cells lost tumor antigen expression (FIG. 10B). Moreover, Atf7ip inhibition significantly increased antigen expression in immune escaped tumor cells (FIG. 10C-E). These results indicate that in two distinct antigen models, Atf7ip deficiency upregulates tumor antigen expression.

### EXAMPLE 4

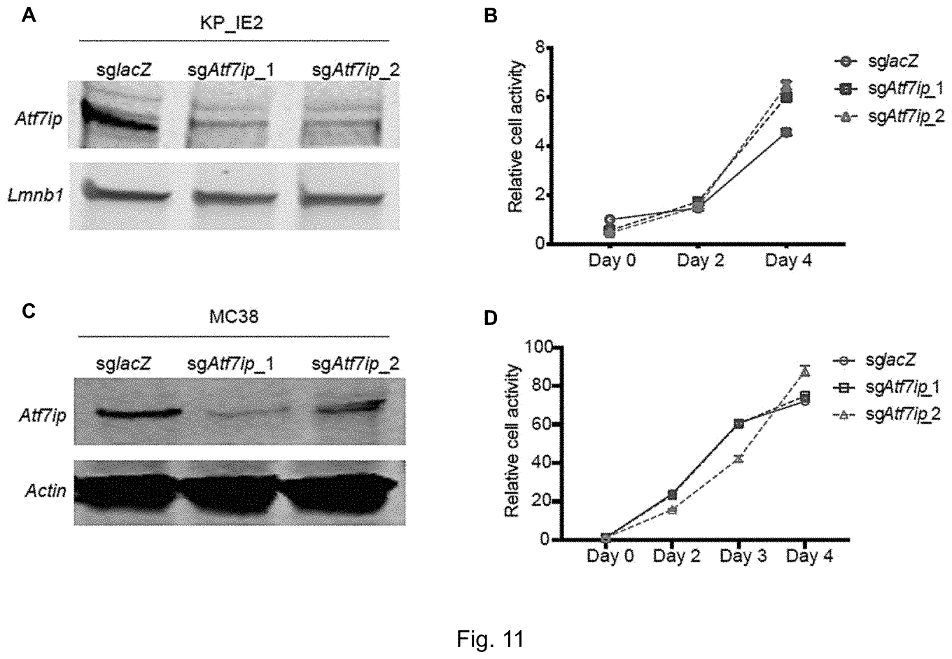

Atf7ip deficiency augments tumor immunogenicity. To further examine if Atf7ip deficiency can restore the immunogenicity of KP-IE2 cells, Atf7ip wild type and Atf7ip deficient KP-IE2 cells were injected into both B6/J immunocompetent mice and NU/NU (nude) immunodeficient mice. Compared to Atf7ip wild type tumors, Atf7ip deficient tumors grew significantly slower in B6/J mice, contributing to prolonged survival benefit (FIG. 2J-L). However, there was no survival benefit to nude mice grafted with Atf7ip deficient KP-IE2 cells (FIG. 2M). Importantly, Atf7ip deficient cells didn't show attenuated proliferation in vitro (FIG. 11). These findings indicate that Atf7ip inhibition results in the restoration of immune surveillance in tumor cells in vivo.

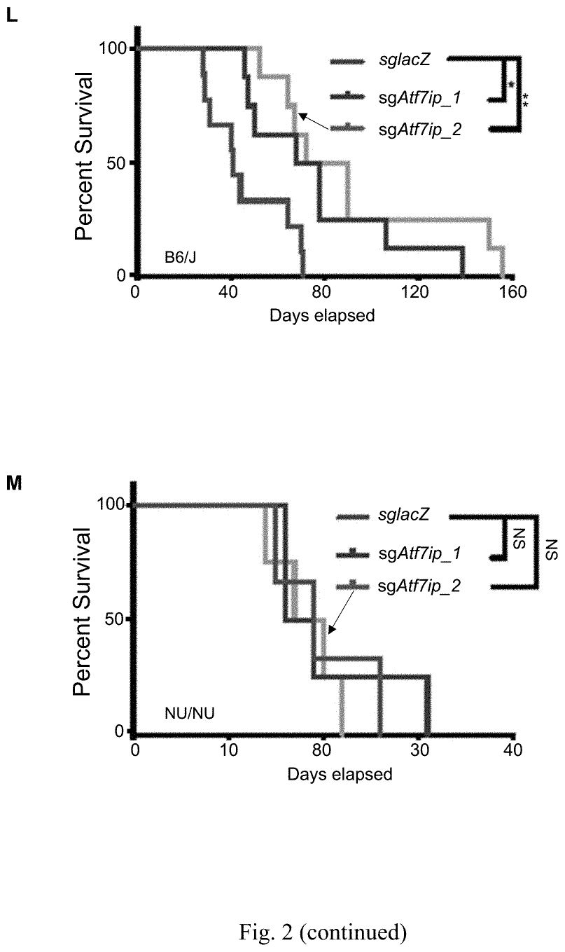

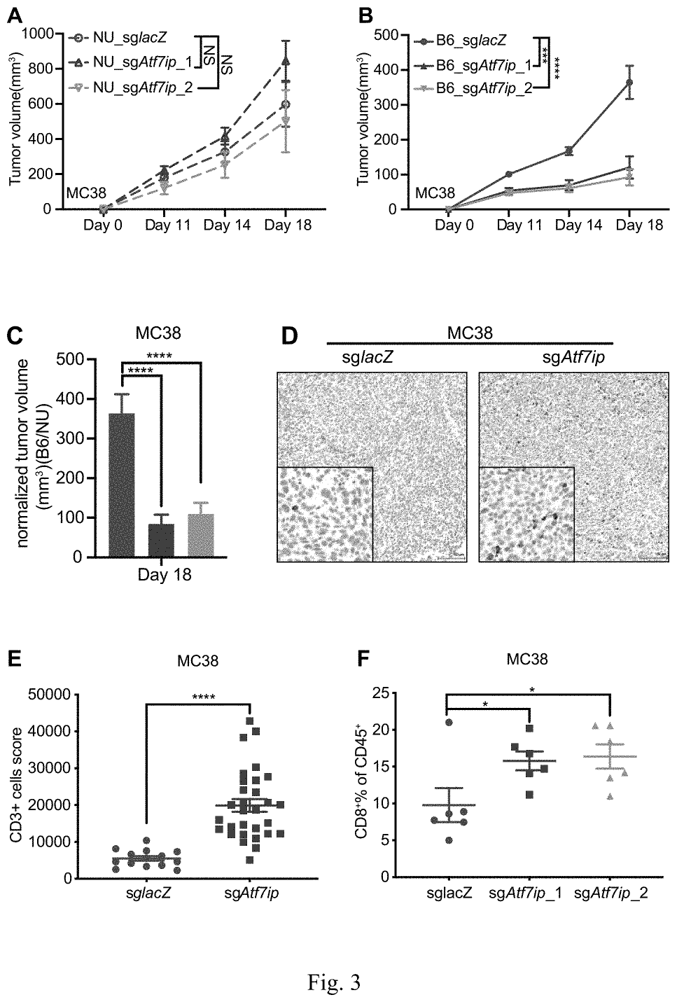

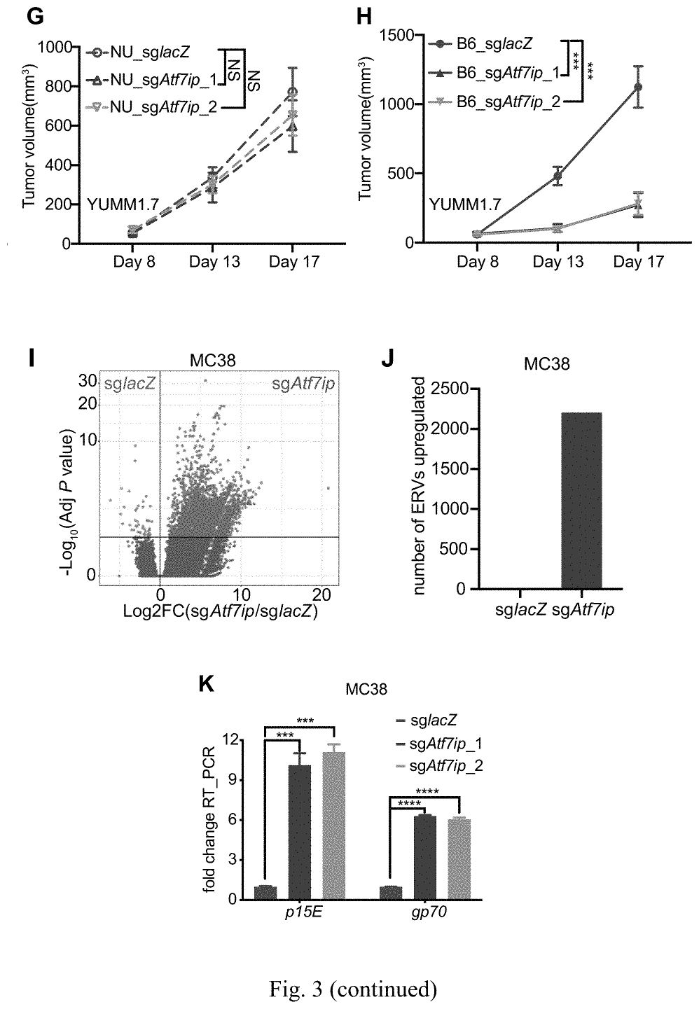

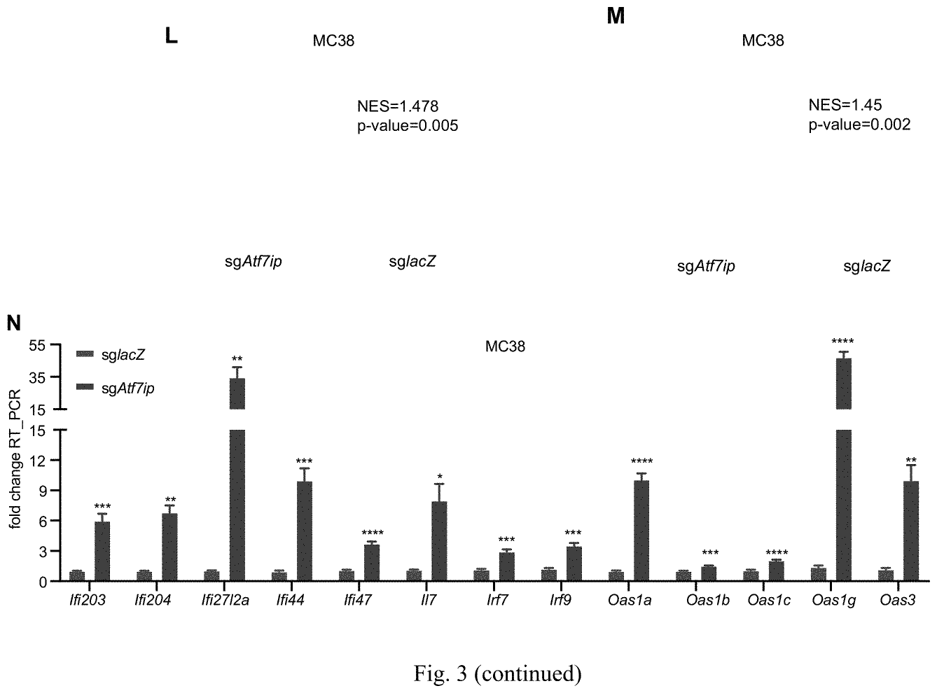

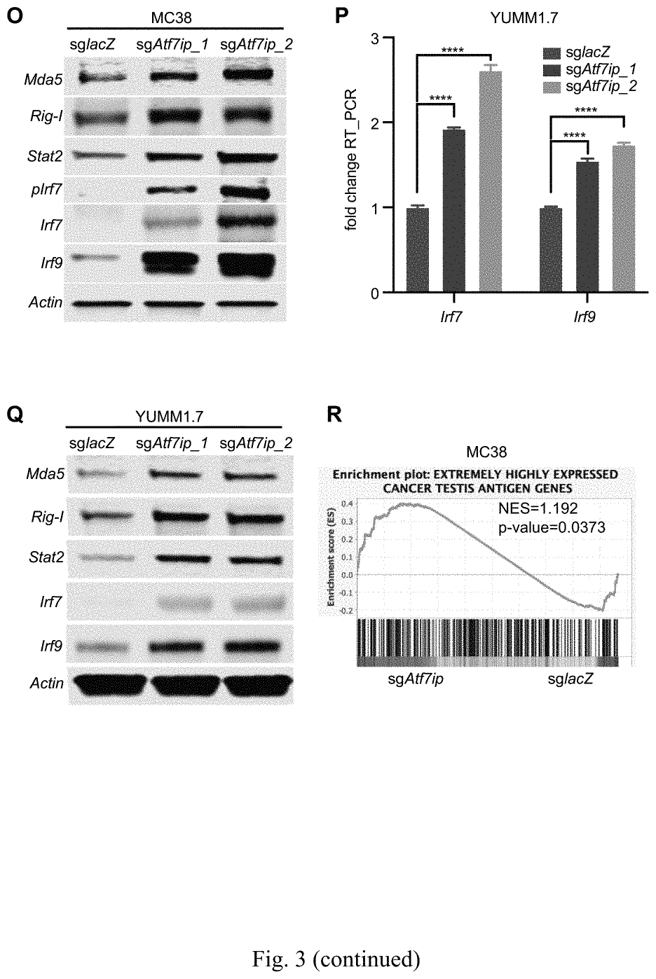

To rule out the possibility that regulation of antigen expression and presentation from Atf7ip was due to an artificial effect from exogenous antigen induction, we expanded this disclosure to analysis of cancer models which have a higher mutation burden (TMB). It was previously shown that high TMB is likely to generate more tumor antigens (Kelderman and Kvistborg, 2016). Here we took advantage of murine colorectal cancer cell line MC38, which has high TMB and is immune-edited (Zhong et al., 2020). Atf7ip deficient MC38 cells also didn't show attenuated proliferation in vitro (FIG. 11). MC38 cells with or without Atf7ip deficiency were subcutaneously injected into both nude mice and B6/J mice. Tumor progression with Atf7ip deficient MC38 cells was significantly inhibited in B6/J mice but not in nude mice (FIG. 3A, B, C). These data indicates that Atf7ip deficiency stimulated an anti-tumor immune response.

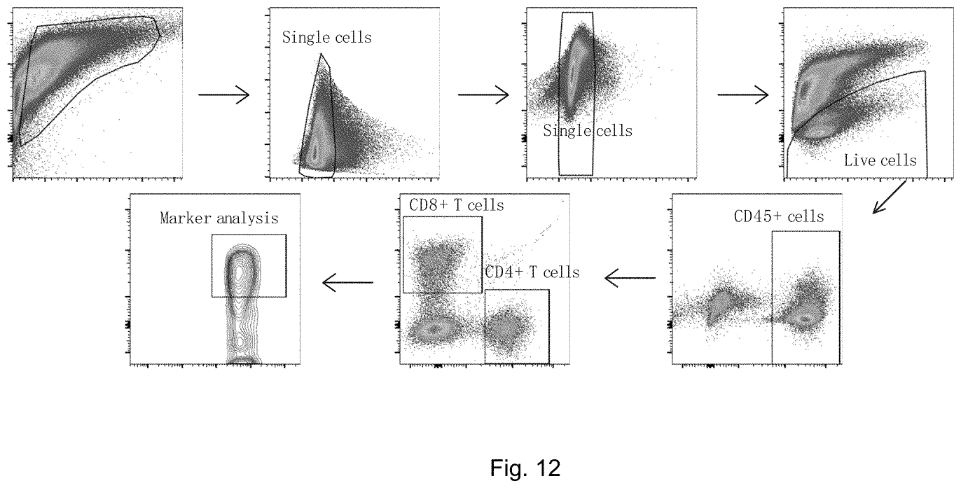

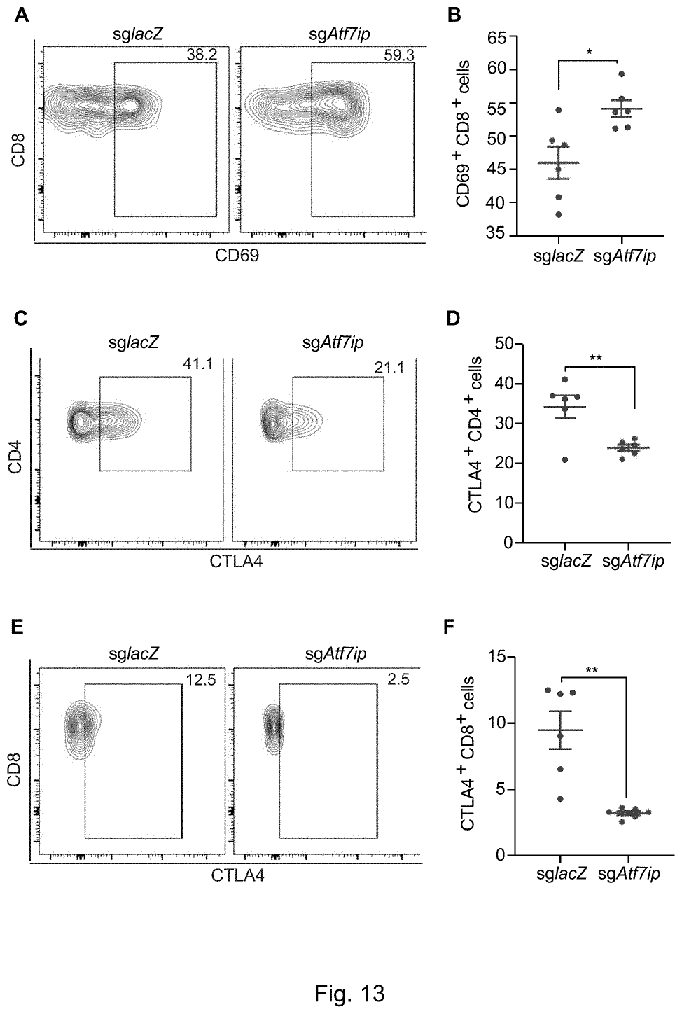

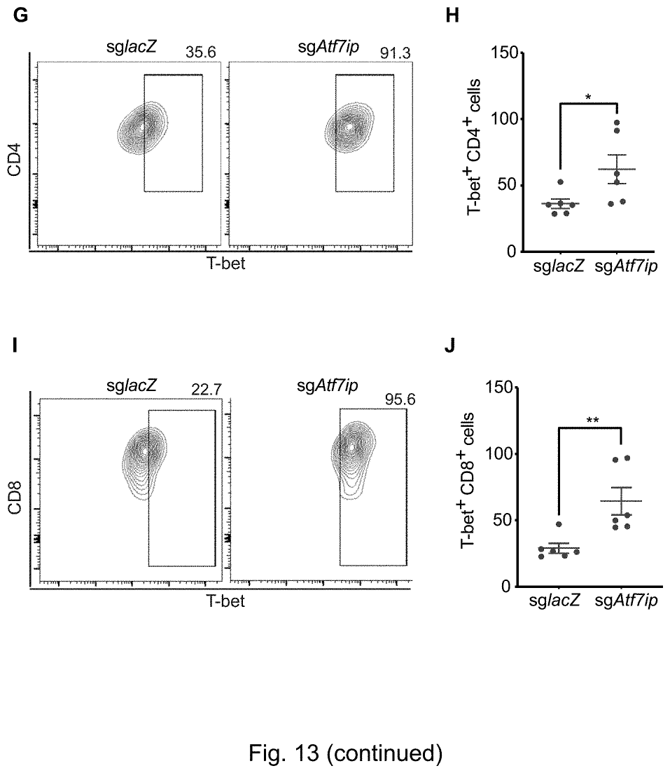

Indeed, immunohistochemistry staining showed that there was a significant increase in the infiltration of CD3+ T cells in Atf7ip deficient tumors (FIG. 3D, E), which further supports that tumor cell-intrinsic Atf7ip deficiency enhances anti-tumor immune response. Consistently, immune profiling analysis showed a significant increase in infiltrated CD8+ T cells in Atf7ip deficient tumors but marginal increase in infiltrated CD4+ T cells (FIG. 3F, FIG. 12). Moreover, the activation of cytotoxic CD8+ T cells was markedly enhanced in Atf7ip deficient tumors (FIG. 13A, B) and infiltrated CD4+ and CD8+ T cells were less exhausted (FIG. 13C-F). T-bet is a key player in generation of type 1 immunity in both T helper and T cytotoxic cells and it is also a transcription factor which promotes the activation in CD8+ effector function (Sullivan et al., 2003). T-bet+ infiltrated CD4+ and CD8+ T cells were significantly increased in Atf7ip deficient tumors (FIG. 13G-J), also suggesting enhanced anti-tumor immunity. Collectively, these results indicate Atf7ip inhibition elevates tumor immunogenicity and the infiltration of activated T cells.

### EXAMPLE 5

Atf7ip deficiency upregulates endogenous tumor antigen expression and boosts anti-tumor immune response. Endogenous retroviral antigens (ERVs) have been found in human tumors (Kassiotis, 2014). Studies have shown the presence of T cell repertoire specifically recognizing ERVs (Kvistborg et al., 2012) and ERVs can potentially serve as immunotherapy targets (Takahashi et al., 2008). In addition, ERVs are potent tumor rejection antigens for some malignancies (Chiappinelli et al., 2015; Kelderman and Kvistborg, 2016; Sheng et al., 2018). Atf7ip was identified as one of the determinants for provirus silencing in embryonic stem cells (Yang et al., 2015). Thus, we propose that Atf7ip deficiency could boost anti-tumor immunity via upregulating the expression of ERVs. To test this hypothesis, we performed RNA sequencing in both Atf7ip wild type and Atf7ip deficient MC38 cells; and monitored the change in ERVs expression upon Atf7ip inhibition. Analysis of ERVs indicated that Atf7ip deficiency significantly upregulated global endogenous retroviral antigens expression (FIG. 3I, J). Specifically, the expression of endogenous retroviral antigens gp70 and p15E, were significantly increased upon Atf7ip inhibition (FIG. 3K). These ERVs are used as vaccines for protecting mice from tumor challenge (Bronte et al., 2003; Kershaw et al., 2001). These results support Atf7ip inhibition promotes ERVs expression.

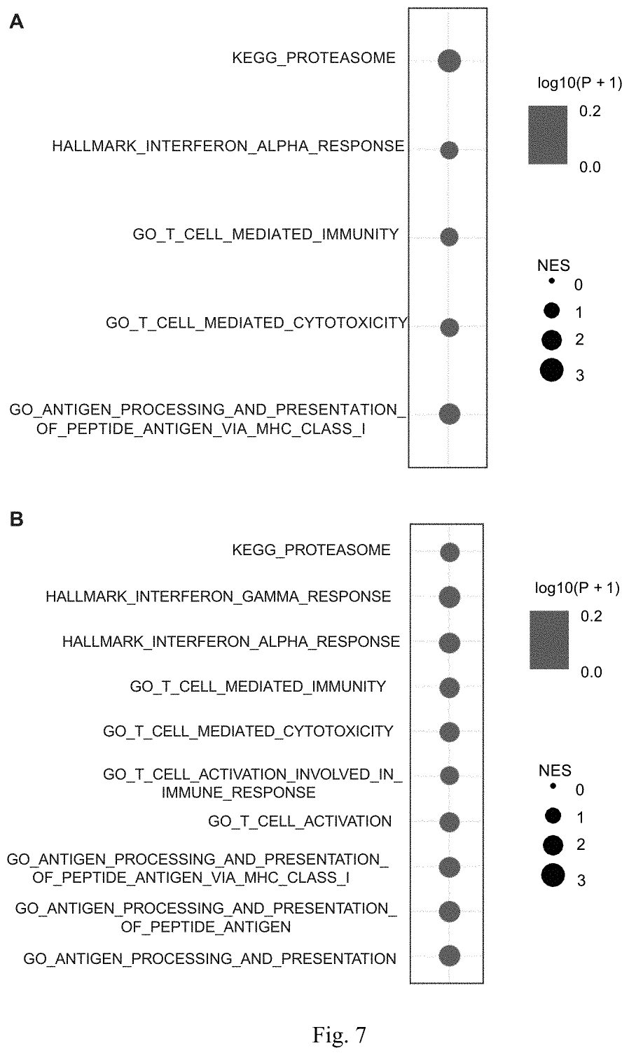

Double strand RNAs (dsRNAs) can be sensed by immune system, resulting in an interferon immune response (Sheng et al., 2018). Indeed, we found that upregulation of ERVs expression in Atf7ip deficient MC38 cells further activated interferon immune response (FIG. 3L, K). Key players in interferon signaling pathways (viral defense), including Irf7 and Irf9, were upregulated in Atf7ip deficient MC38 cells at both a transcriptional and translational level (FIG. 3N, O). We also observed an enrichment of several anti-tumor immunity related signaling pathways in ATF7IP low expressed tumor samples in the TCGA pan-cancer dataset (FIG. 7).

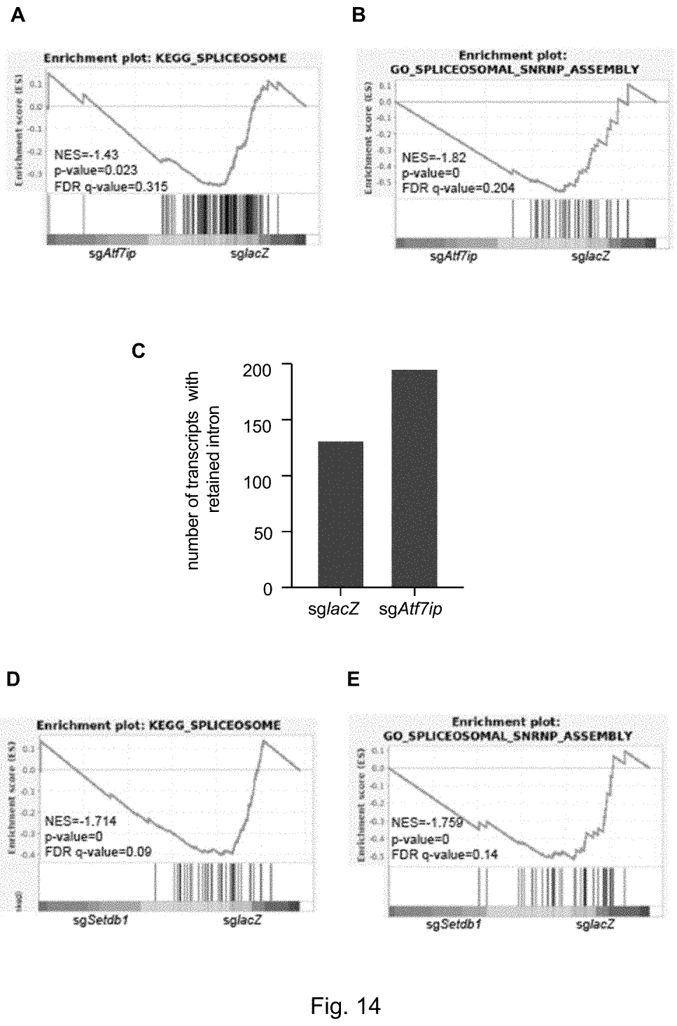

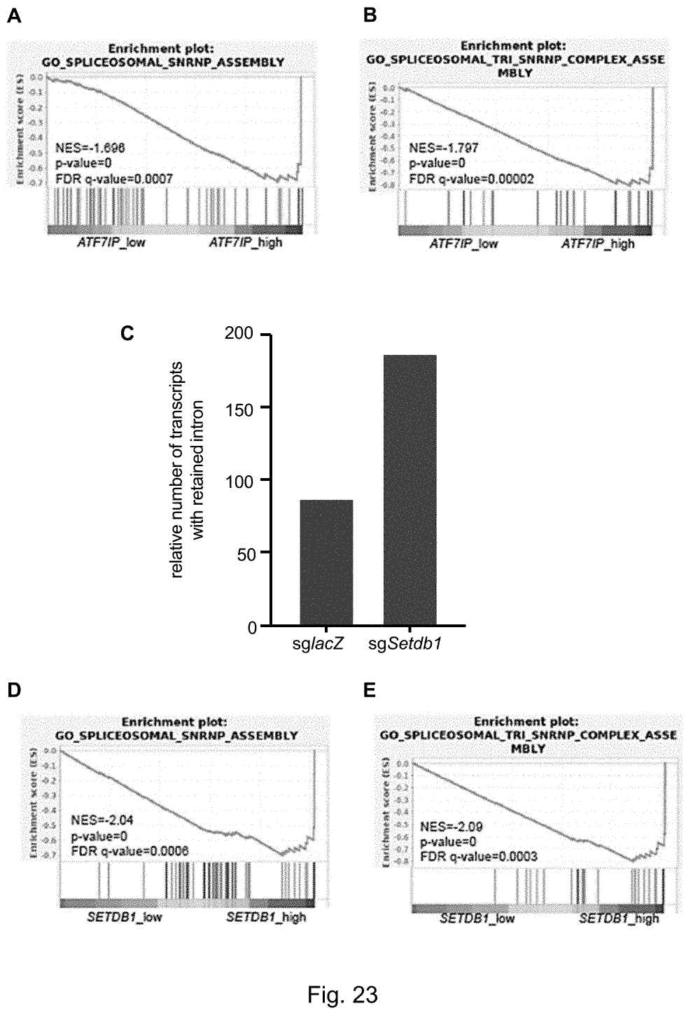

Several types of endogenous tumor antigens other than ERVs exist (Kelderman and Kvistborg, 2016), and thus we investigated if Atf7ip deficiency could cause changes to additional endogenous tumor antigens in tumor cells. For examples, cancer testis antigens (CTs) are another type of tumor endogenous antigens that have been applied in clinical trials for immunotherapy (Hunder et al., 2008; Robbins et al., 2015). Moreover, novel cancer vaccines targeting various CTs have been developed among multiple malignancies and many of them have advanced to clinical trials with impressive efficacy (Aruga et al., 2014; Kono et al., 2012). GSEA analysis indicated global upregulation of CTs upon Atf7ip inhibition (Wang et al., 2016) (FIG. 3R). In addition, the change in alternative splicing may produce more splicing-derived neoepitopes (Frankiw et al., 2019; Park and Chung, 2019). Tumor-specific retained intron neoepitopes have been identified in both patient cancer and cell line derived samples (Smart et al., 2018). Our GSEA analysis indicates Atf7ip deficiency inhibited spliceosome activity (FIG. 14A, B). There was a significant increase in RNA intron retention events in Atf7ip deficient MC38 cells (FIG. 14C). We also observed the downregulation of spliceosome assembly activities in Atf7ip low expressed tumor samples in the TCGA lung adenocarcinoma dataset (FIG. 23A, B). Collectively, these results indicate that Atf7ip inhibition promotes tumor antigen expression and stimulates anti-tumor immunity.

We further expanded this disclosure to another tumor model YUMM1.7 (Yale University Mouse Melanoma) (Meeth et al., 2016). YUMM1.7 well recapitulates human melanoma because it has BRAFV600E driver mutation, inactivating CDKN2A and PTEN mutations (Meeth et al., 2016). YUMM1.7 cells with or without Atf7ip deficiency were subcutaneously injected into both nude mice and B6/J mice. Tumor progression with Atf7ip deficient YUMM1.7 cells was significantly inhibited in B6/J mice but not in nude mice (FIG. 3G, H). Irf7 and Irf9 were upregulated in Atf7ip deficient YUMM1.7 cells (FIG. 3P, Q). These data indicates that Atf7ip deficiency stimulated anti-tumor immune response.

### EXAMPLE 6

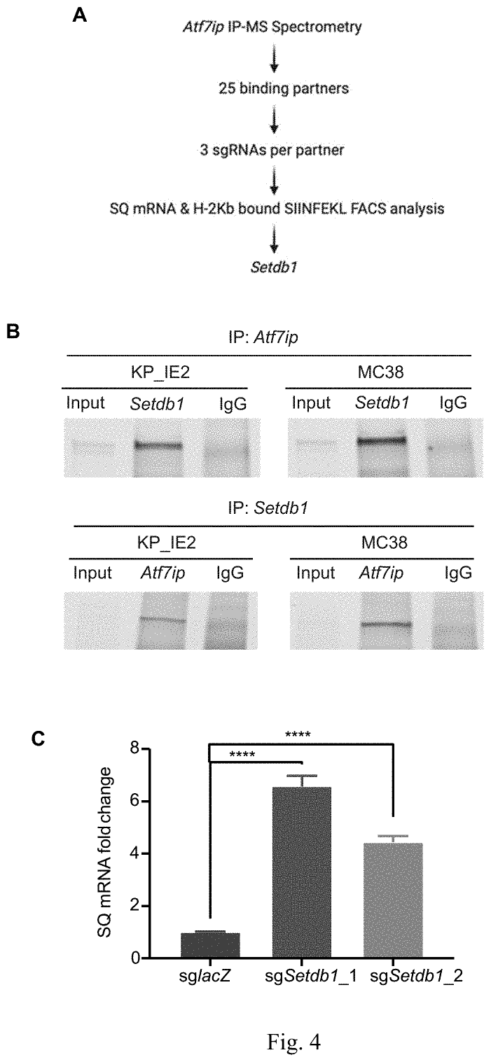

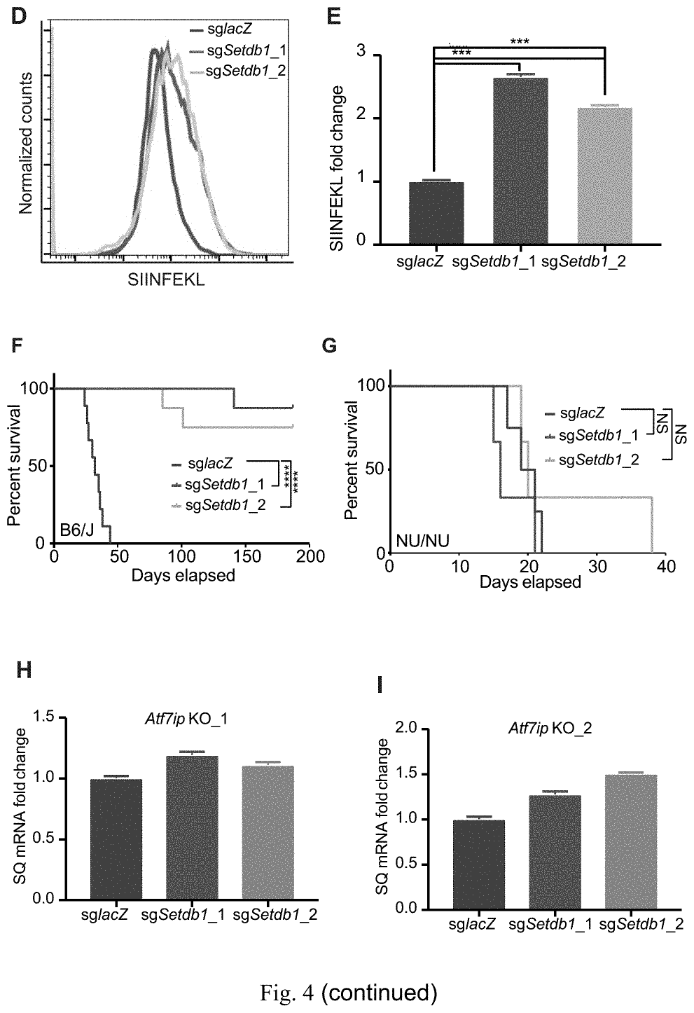

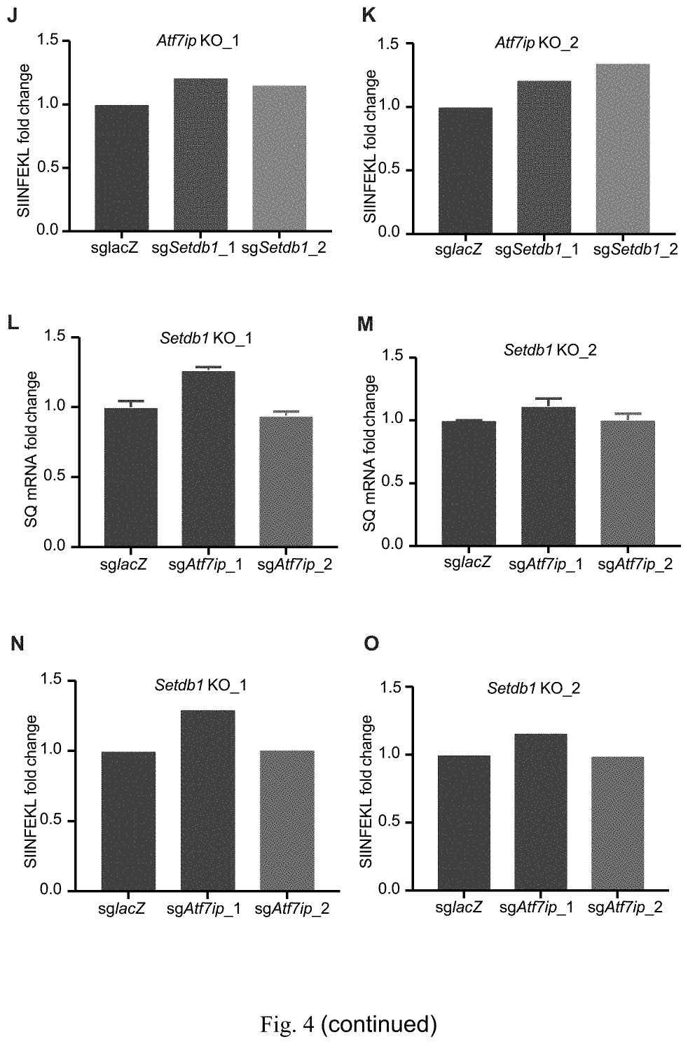

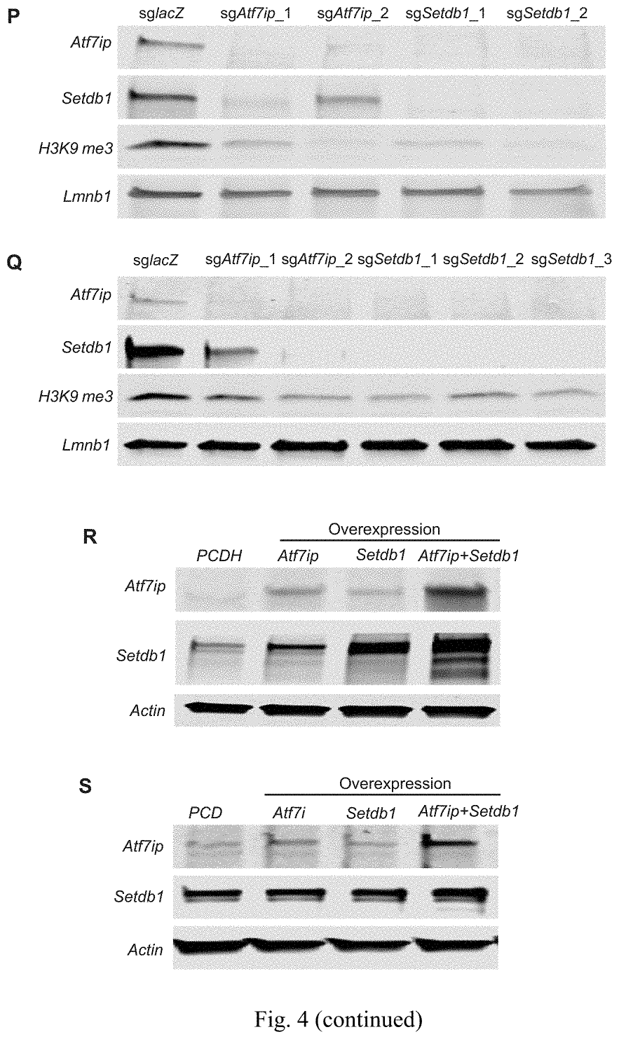

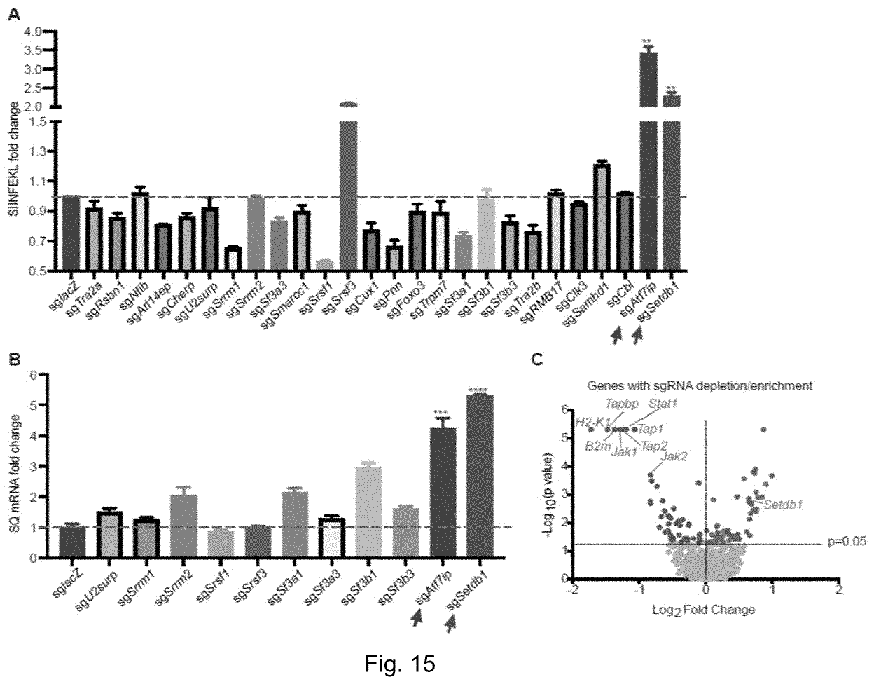

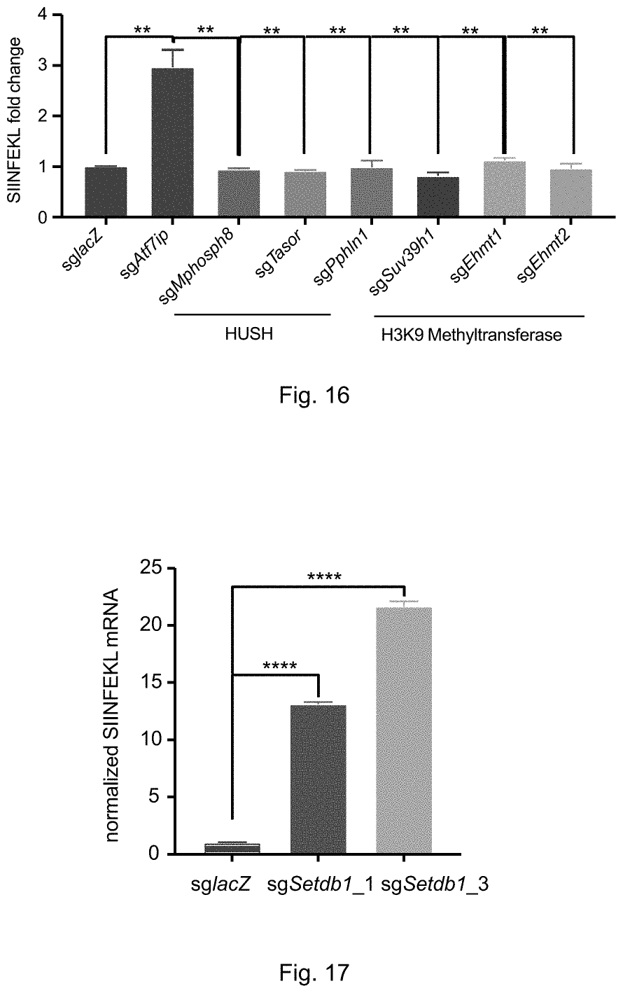

Setdb1 acts as a partner gene of Atf7ip in the regulation of tumor antigen expression. To explore other key players that act alongside Atf7ip in regulating tumor antigen expression and presentation, we performed immunoprecipitation mass-spectrometry of Atf7ip (IP-MS). Following this, we picked up 25 Atf7ip binding partners and investigated if their inhibition could modulate SQ transcription or presentation (FIG. 4A). Setdb1 was the only partner that not only interacts with Atf7ip but also regulates SQ expression and presentation (FIG. 4B-E; FIG. 15A, B). Moreover, Setdb1 is one of the candidates whose inhibition promoted SIINFEKL (SEQ ID NO:47) expression and presentation in our initial screen (FIG. 15C). Setdb1 is one of the histone H3K9 methyltransferases. HUSH complex was identified to work with Setdb1 for transcriptional silencing (Tchasovnikarova et al., 2015). However, the regulation of SQ presentation is independent of HUSH complex and other H3K9 methyltransferases (FIG. 16). In addition, Setdb1 inhibition significantly increased SIINFEKL (SEQ ID NO:47) expression in our KP-LucOS immune escaped tumor model (FIG. 17). These results indicate that Setdb1 deficiency can also upregulate antigen expression.

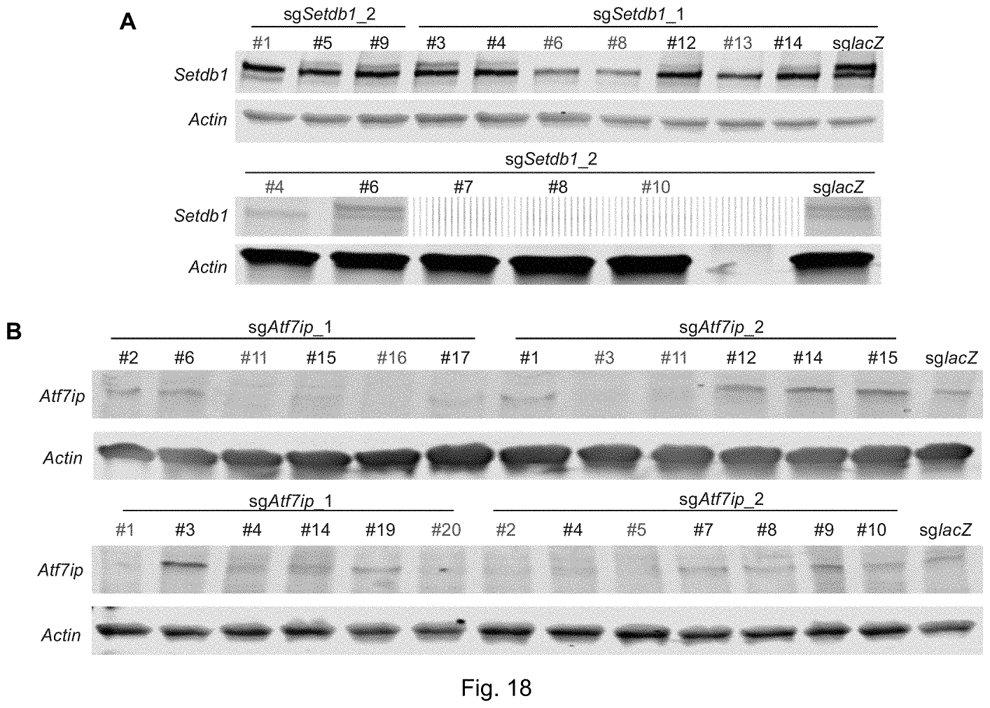

To test whether Setdb1 deficiency can restore the immunogenicity of KP-IE2 cells, KP-IE2 cells with or without Setdb1 inhibition were injected into both B6/J mice and nude mice (FIG. 18A). Setdb1 deficient tumors grew significantly slower in B6/J mice, leading to significantly prolonged survival benefit (FIG. 4F). However, we found no survival benefit in nude mice grafted with Setdb1 deficient KP-IE2 cells (FIG. 4G). These results indicate that Setdb1 inhibition resulted in the restoration of immune surveillance to KP-IE2 cells in vivo. To understand if there is a synergistic effect on regulating antigen expression and presentation when inhibiting both Atf7ip and Setdb1, we generated Atf7ip and Setdb1 double knocked-out KP-IE2 cell lines. Atf7ip and Setdb1 double inhibition showed no synergistic effect on SQ transcription and presentation (FIG. 4H-O), which further supports the idea that they work in the same pathway, and knocking out either one achieves similar consequences. It has been reported that Atf7ip is essential for Setdb1 stability (Timms et al., 2016). Consistently, Atf7ip and Setdb1 proteins were positively correlated with each other in both KP-IE2 and MC38 cell lines (FIG. 4P, Q). Furthermore, overexpressing Atf7ip increased Setdb1 protein level and overexpressing Setdb1 increased Atf7ip protein level in KP-IE2 cell line (FIG. 4R). The elevation of their protein levels was further enhanced when both Atf7ip and Setdb1 were overexpressed together in both KP-IE2 and MC38 cell lines (FIG. 4R, S). Thus, it is plausible to hypothesis that Atf7ip and Setdb1 work in a complex to control tumor antigen expression and presentation.

### EXAMPLE 7

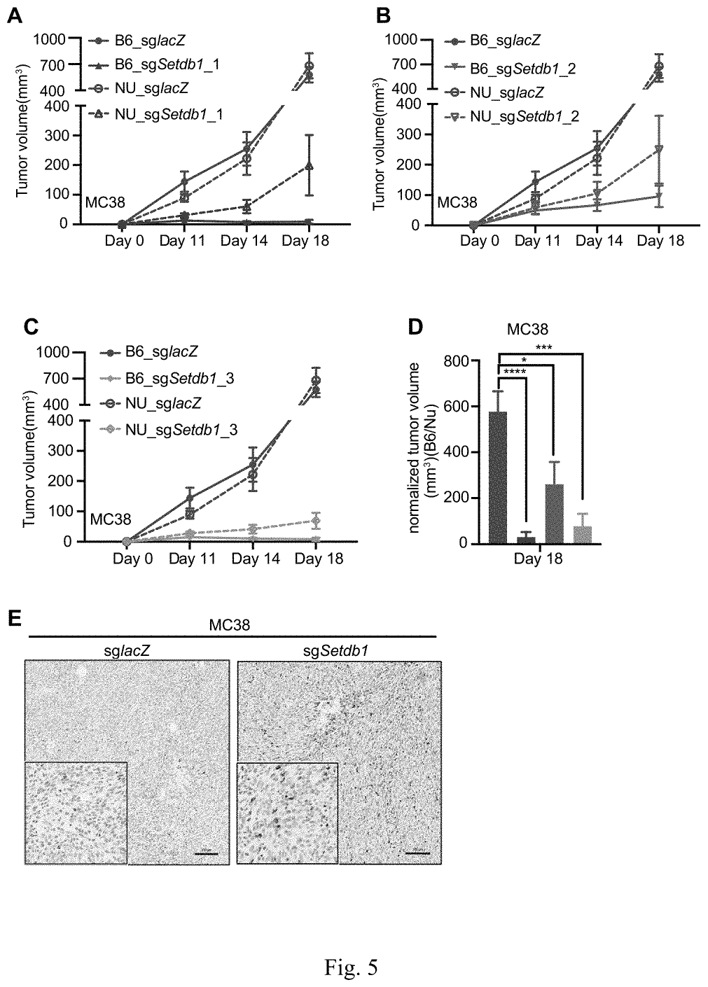

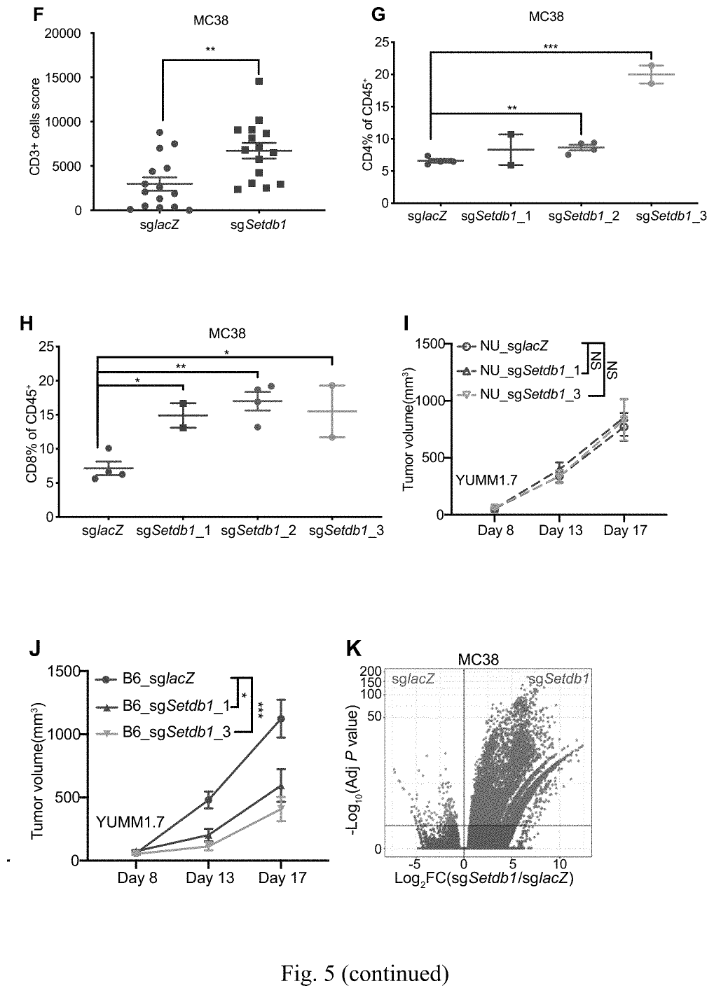

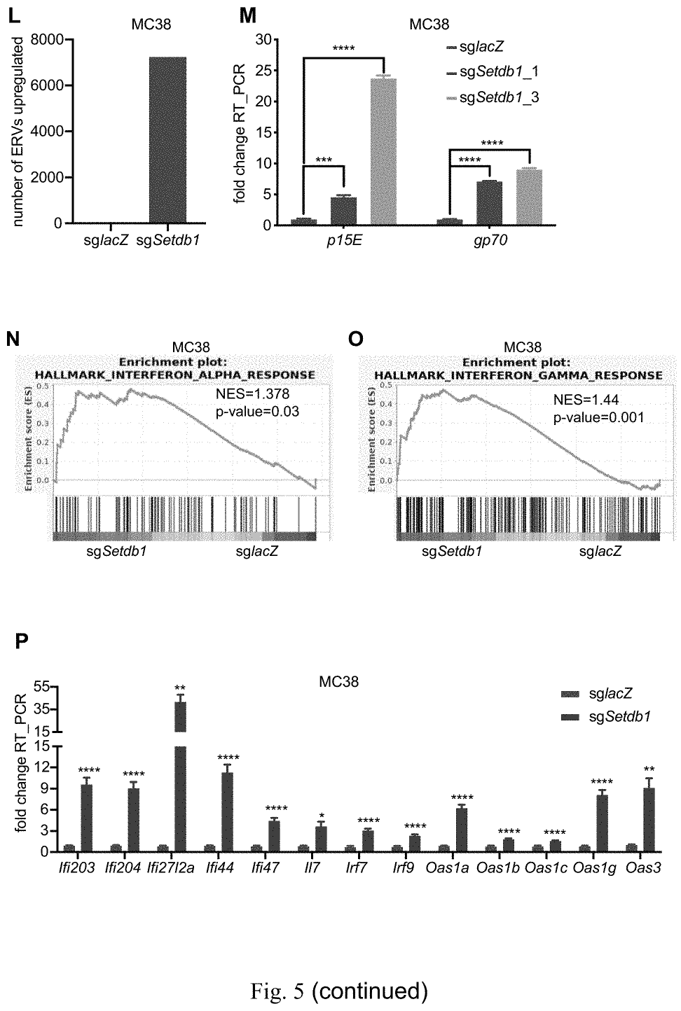

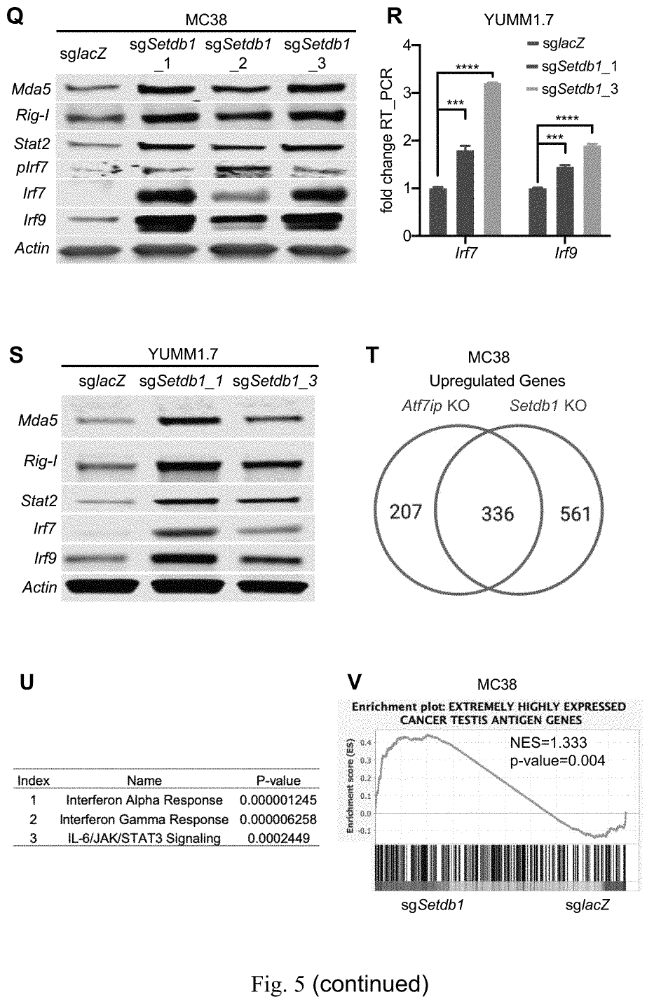

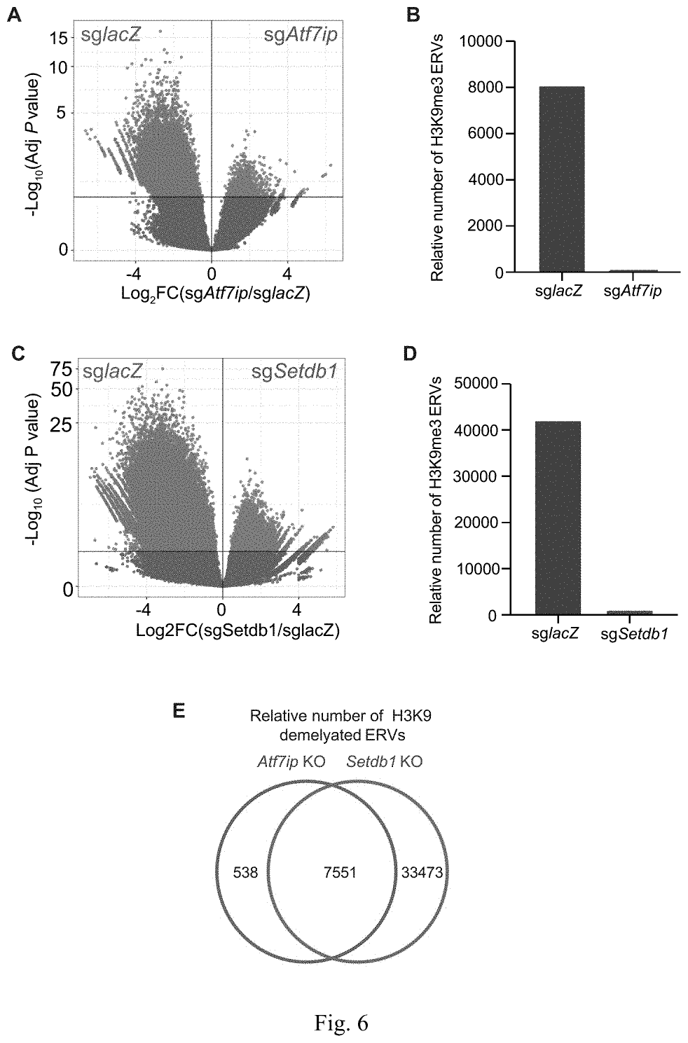

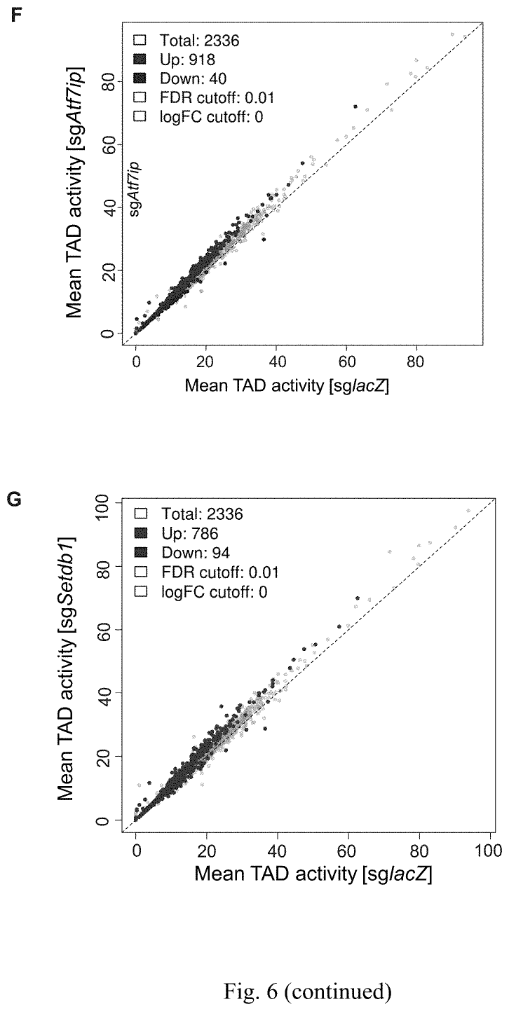

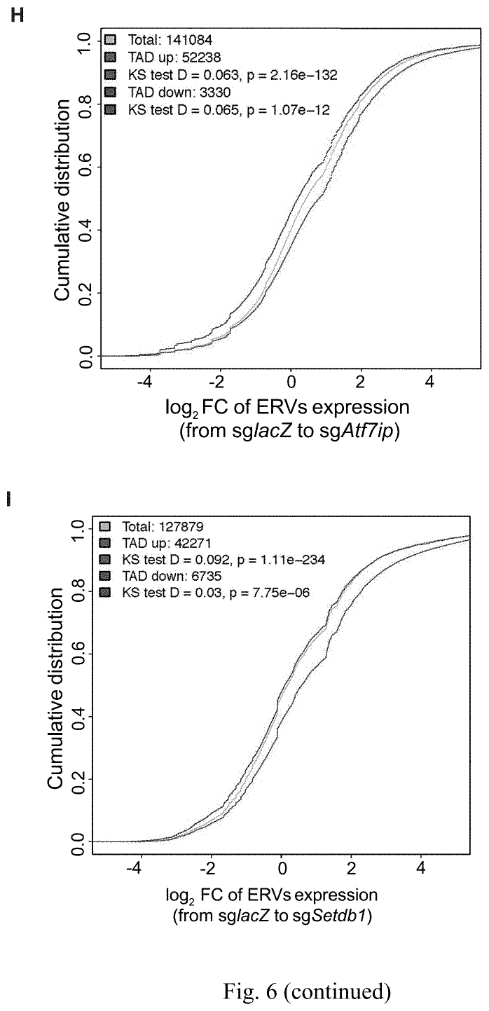

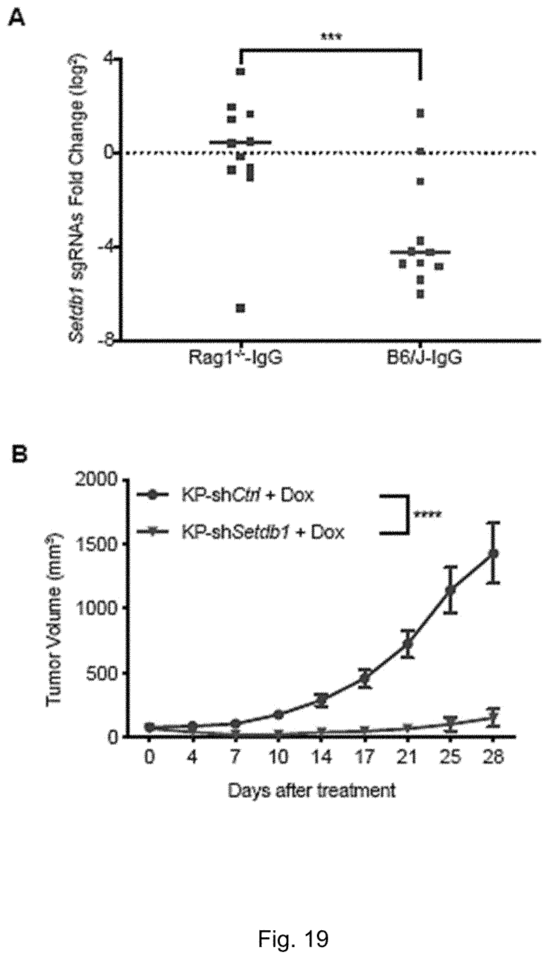

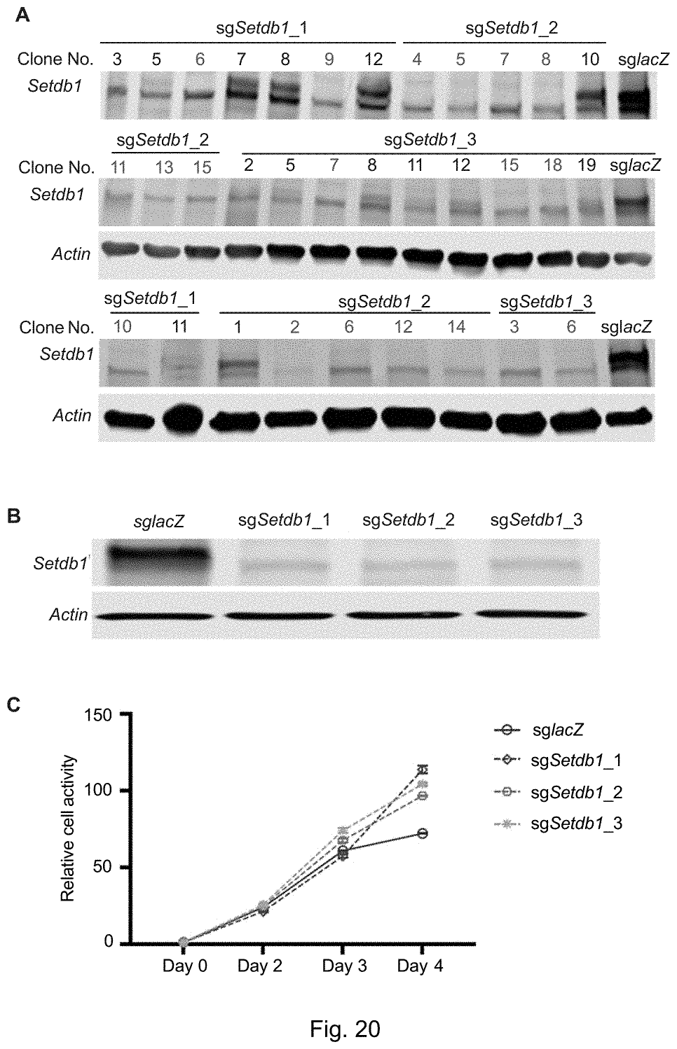

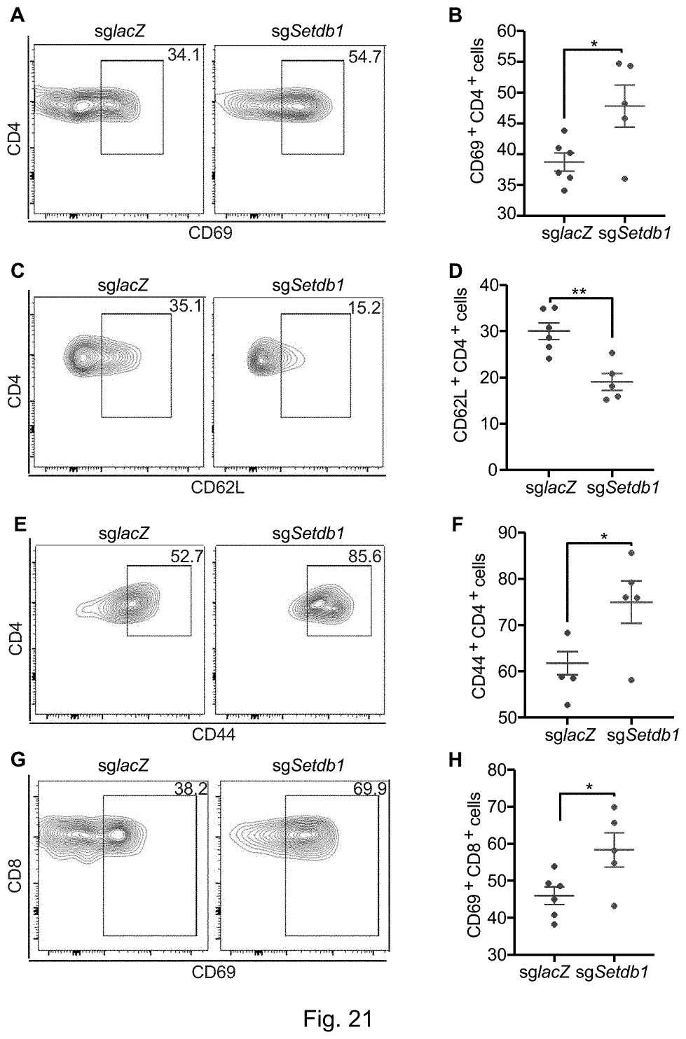

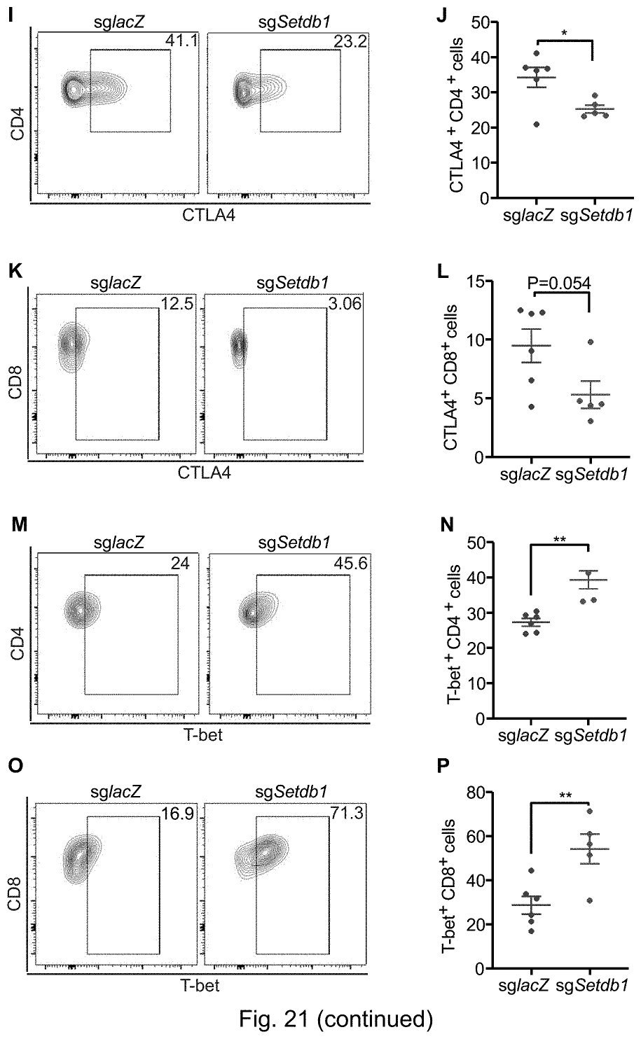

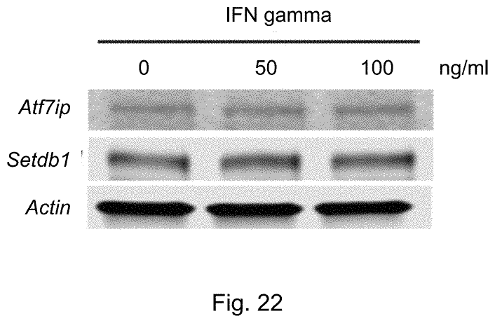

Setdb1 deficiency also boosts anti-tumor immune response via increasing tumor antigen expression. In our previous in vivo epigenetic CRISPR screen, Setdb1 was identified as an epigenetic target that, upon loss, modulated the antitumor immune response in B6/J mice (Li et al., 2020) (FIG. 19A, B). To investigate if Setdb1 inhibition could also stimulate anti-tumor immunity in different tumor models, we established Setdb1 depleted MC38 cells (FIG. 20A-C). MC38 cells and Setdb1 deficient MC38 cells were subcutaneously injected into both nude mice and B6/J mice. Greater inhibition in tumor growth was found in Setdb1 deficient MC38 tumor growth in B6/J mice compared to that in nude mice, indicating that Setdb1 deficiency also induced an anti-tumor immune response (FIG. 5A-D). Indeed, there was a significant increase in infiltrated CD3+ T cells in Setdb1 deficient tumors as assessed by IHC staining (FIG. 5E, F). Immune profiling experiments also indicated an increase in infiltration of both CD4+ and CD8+ T cells in Setdb1 deficient tumors (FIG. 5G, H). Moreover, the activation of infiltrated T cells was markedly enhanced in Setdb1 deficient tumors (FIG. 21A-H) and infiltrated T cells were less exhausted (FIG. 21I-L). T-bet+ infiltrated T cells were significantly increased in Setdb1 deficient tumors, which also indicates enhanced anti-tumor immunity (FIG. 21M-P).

In order to analyze changes in expression of ERVs upon Setdb1 inhibition, we performed RNA sequencing for both Setdb1 wild type and Setb1 deficient MC38 cells. ERVs analysis indicated that Setdb1 deficiency significantly upregulated global endogenous retroviral antigens expression (FIG. 5K, L). The expression of endogenous retroviral antigen gp70 and p15E were significantly increased upon Setdb1 inhibition (FIG. 5M).

Moreover, upregulation of the expression of ERVs further activated interferon immune response (FIG. 5N, O). Key players in interferon signaling pathways (viral defense), including Irf7 and Irf9, were upregulated in Setdb1 deficient MC38 cells, as demonstrated by their transcriptional and translational level (FIG. 5P, Q). We also observed an enrichment of several anti-tumor immunity related signaling pathways in SETDB1 low expressed tumor samples in the TCGA pan-cancer dataset (FIG. 7). In addition, loss of Setdb1 and Atf7ip shared a similar impact on transcriptome (FIG. 5T). Enrichr analysis showed that the top three upregulated signaling pathways upon loss of Setdb1 and Atf7ip are interferon signaling pathways (FIG. 5U).

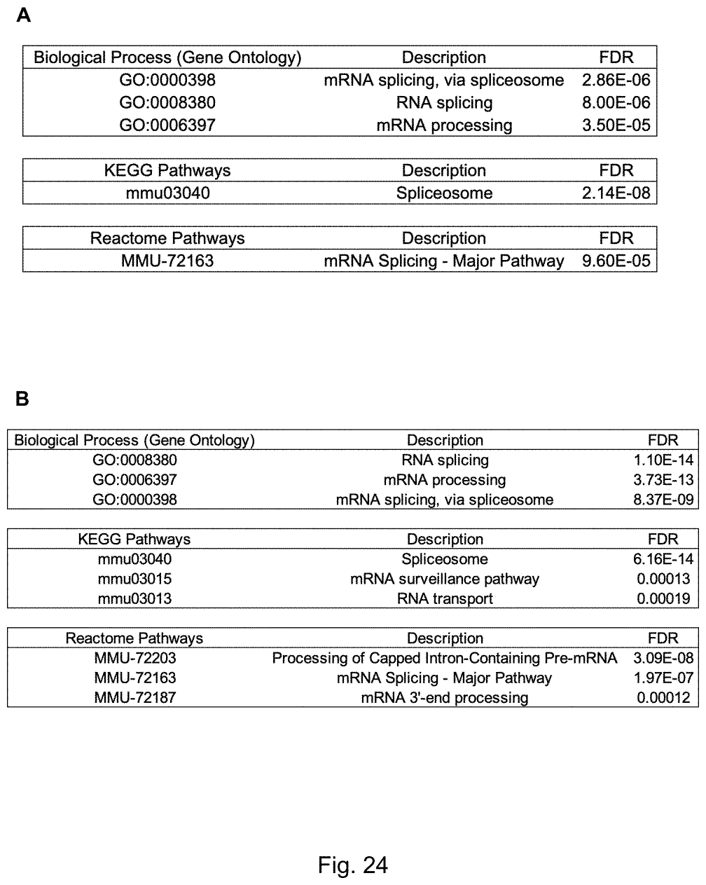

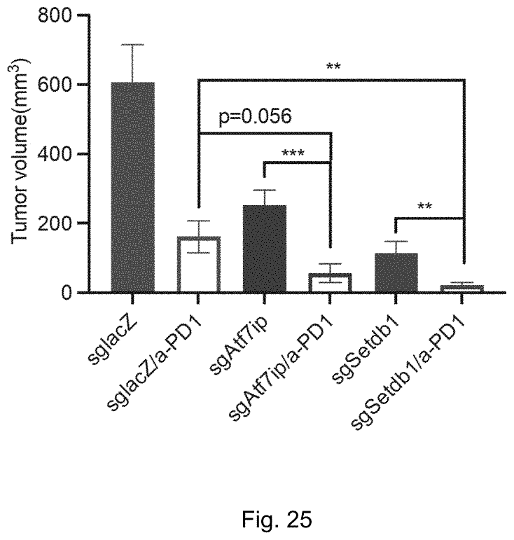

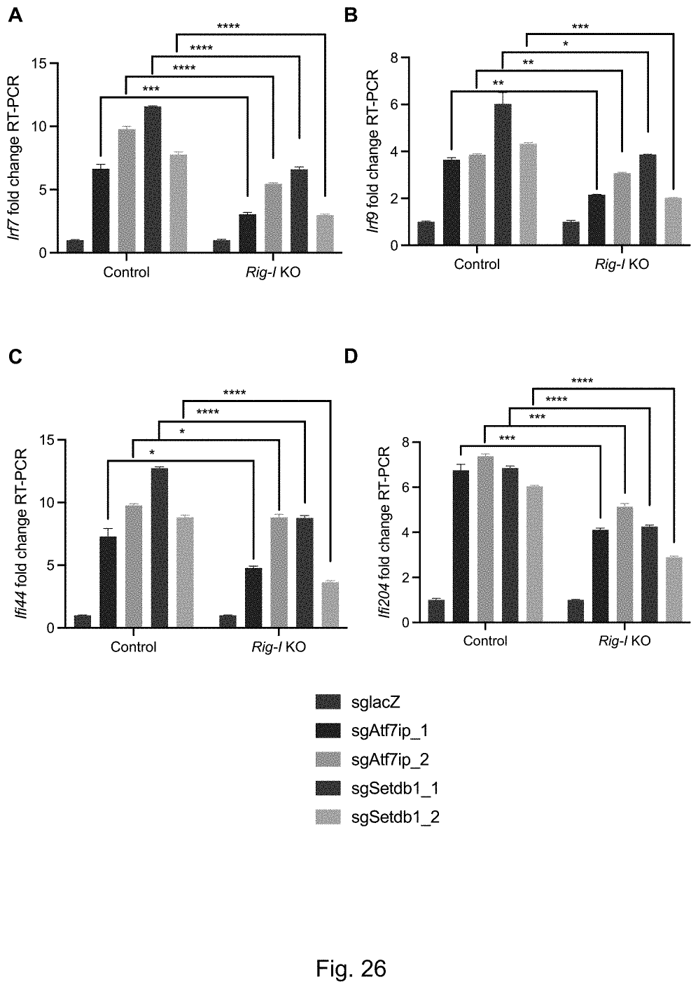

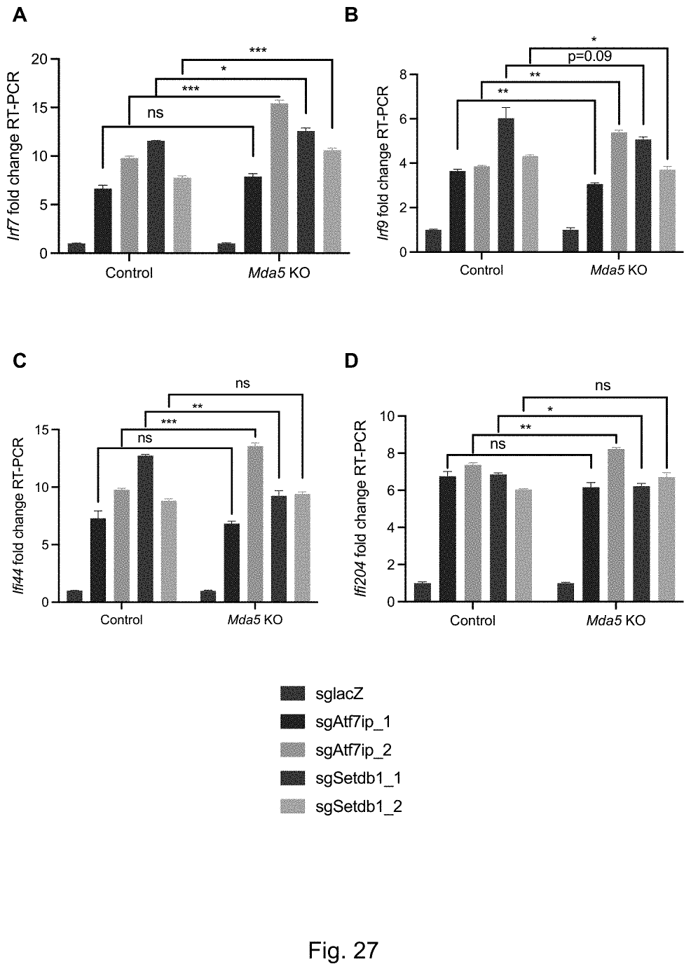

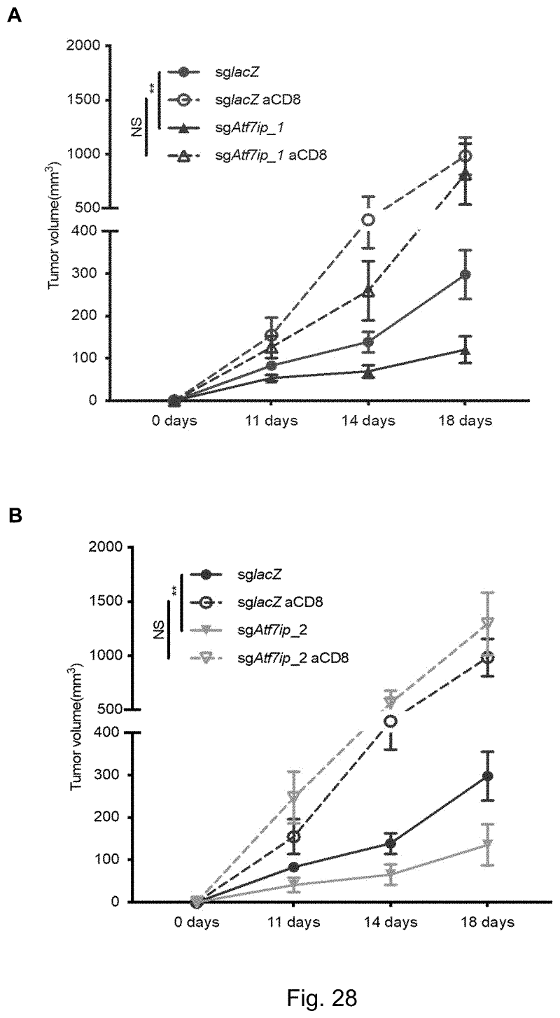

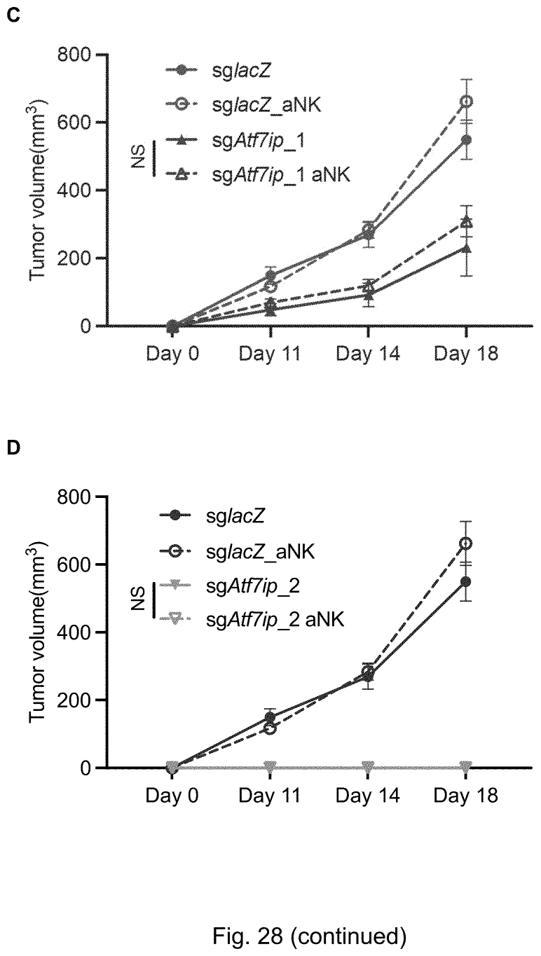

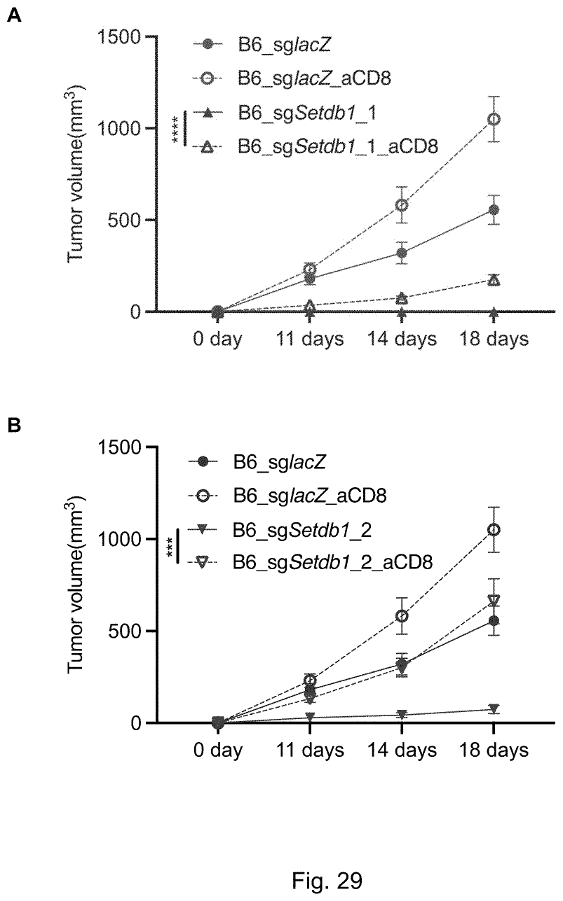

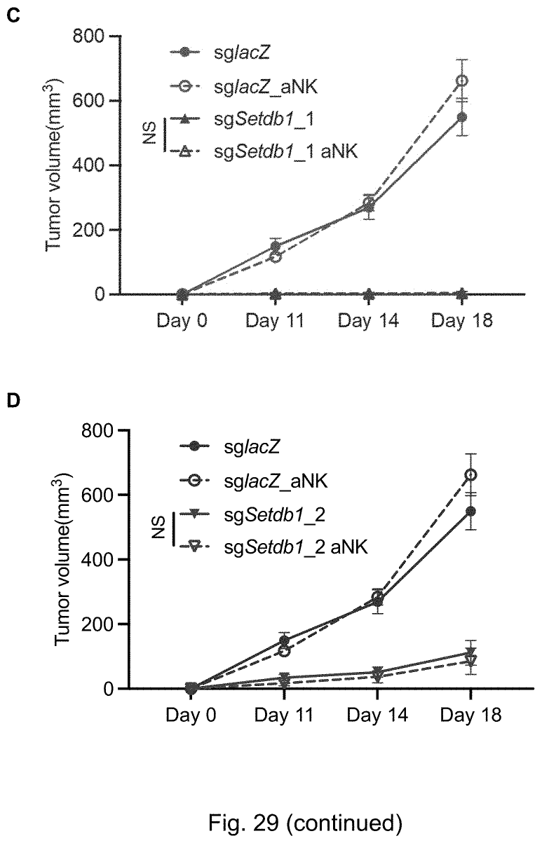

Furthermore, GSEA analysis also indicates global upregulation of CTs upon Setdb1 inhibition (FIG. 5V), which phenocopied Atf7ip inhibition. GSEA analysis also indicates Setdb1 deficiency inhibited spliceosome activity (FIG. 14D, E). There was also a significant increase in intron retention events in Setdb1 deficient MC38 cells (FIG. 23C). This extensive splicing changes affecting retained introns also happened in additional two human lung cancer cell lines H1299 and H226 upon knocking out Atf7ip. Moreover, we observed the downregulation of spliceosome assembly activities in Setdb1 low expressed tumor samples in the TCGA lung adenocarcinoma dataset (FIG. 23D, E). This abnormal spliceosome activity and mRNA alternative splicing could be due to the change of Atf7ip-Setdb1 network. The analysis of Atf7ip IP-MS by STRING database indicates a significant number of Atf7ip binding partners play a role in mRNA alternative splicing (FIG. 24A, B). These results indicate Setdb1 inhibition promotes tumor antigen expression and stimulates anti-tumor immunity.

We further expanded this disclosure to the tumor model YUMM1.7. YUMM1.7 cells with or without Setdb1 deficiency were subcutaneously injected into both nude mice and B6/J mice. Tumor progression with Setdb1 deficient YUMM1.7 cells was significantly inhibited in B6/J mice but not in nude mice (FIG. 5I, J). Irf7 and Irf9 were upregulated in Setdb1 deficient YUMM1.7 cells (FIG. 5R, S). These data indicates that Setdb1 deficiency stimulated anti-tumor immune response.

Atf7ip or Setdb1 Overexpression results in Tumor Immune Evasion

The foregoing Examples demonstrate that inducing Atf7ip or Setdb1 deficiency restrains tumor progression through increasing tumor immunogenicity, stimulating anti-tumor immunity and enabling immune surveillance. The following will be recognized by those skilled in the art from this disclosure.

Defects in the presentation of tumor-antigens, such as loss of antigen expression (either at the DNA, RNA, or post-translational level), mediates resistance to immune surveillance (Draghi et al., 2019). In this disclosure we established an immune escaped tumor model with defective antigen presentation and performed an epigenetic CRISPR screen. We identified Atf7ip and Setdb1 as therapeutic targets which can reverse the silencing of tumor antigens and reactivate the immune system to eliminate tumor cells. Thus this disclosure provides therapeutic strategies to overcome tumor immune evasion.

We integrated exogenous antigen SQ sequence to the cancer cell genome. The ectopic expression of antigen SQ in cancer cells activated the cancer immune editing process in vivo. The immune cells selectively killed the cancer cells with high expression of SQ. However, the cancer cells with decreased immunogenicity through silencing antigen SQ expression established tumors after 10 months. Importantly, this model mimicked the process of human cancer evolution (Rosenthal et al., 2019), during which the promoter of genes containing neo-antigenic mutations became hypermethylated, highlighting an epigenetic mechanism of immune escape. Therefore, our model provided a state-of-the-art tool to identify the regulators of tumor antigen expression via an epigenetic CRISPR screen.

Prior to this disclosure, the roles of Atf7ip and Setdb1 in anti-tumor immunity remain elusive. In this disclosure, we systemically investigated their roles in regulating tumor antigen expression and enhancing anti-tumor immune response, and describe their use in cancer immunotherapy.

Endogenous retroviral antigens (ERVs) are a type of cancer specific antigens, which are silenced in normal tissues. ERVs are thought to activate adaptive immunity and contribute to immune response against cancer cells. We observed a significant upregulation in the expression of ERVs upon Atf7ip and Setdb1 inhibition and subsequent an anti-tumor immune response in immunocompetent hosts. Without intending to be bound by any particularly theory, it is considered that this is the first disclsoure to show that Atf7ip and Setdb1 play an important role in modifying tumor immune microenvironment, through promotion of ERVs expression. In addition, the immune profiling showed a significant expansion of T-bet+ infiltrated T cells in Atf7ip and Setdb1 deficient tumors and a remarkedly elevated Th1 anti-tumor immune response.

Through IP-MS of endogenous Atf7ip and RNA sequencing analysis, we have shown for the first time that Atf7ip and Setdb1 potentially interact with RNA splicing machinery in tumor cells. Atf7ip and Setdb1 deficiency modified RNA alternative splicing and created more intron retained RNA. This change in alternative splicing may produce more splicing-derived neoepitopes.

It is interesting that antigen SQ expression and presentation was reversed without significant change in the methylation of its promoter upon Atf7ip inhibition in the immune escaped KP-IE2 cell line (FIG. 2H, I). These results suggest Atf7ip and Setdb1 might function downstream of DNA methylation.

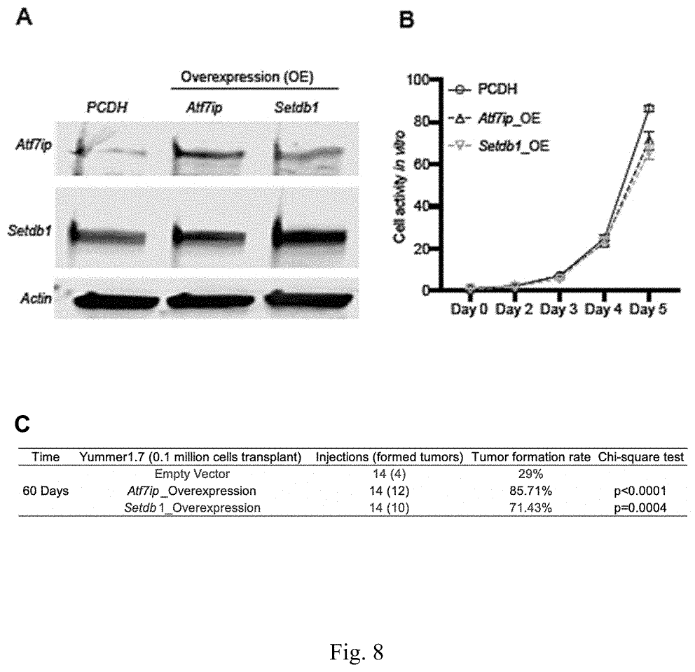

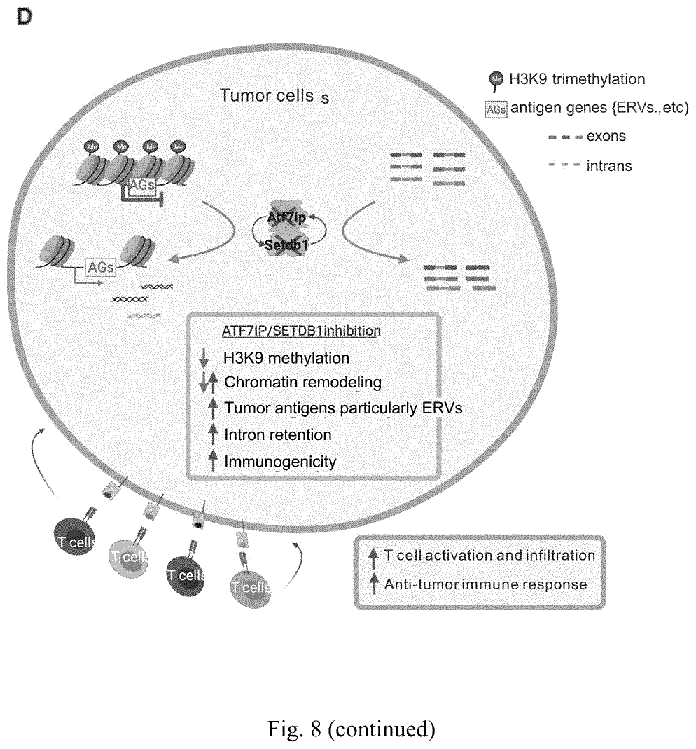

In summary, we performed an epigenome-focused CRISPR screen and identified ATF7IP and SETDB1 as therapeutic targets in stimulating anti-tumor immunity. Functional and mechanistic studies showed that tumor cell-intrinsic Atf7ip or Setdb1 deficiency promotes antigen expression and presentation, especially for endogenous retroviral antigens and intron retained neoepitopes. Upregulation of antigen expression and presentation increases T cell infiltration and activation, leading to enhanced anti-tumor immune response (FIG. 8D). Atf7ip and Setdb1 deficiency exerts significant inhibition on tumor growth in immunocompetent host. Thus, ATF7IP and Setdb1 inhibition can serve as potential novel immunotherapeutic strategies for cancer.

While the present invention has been described through various embodiments, routine modifications will be apparent to those skilled in the art, which modifications are intended to be included within the scope of the present disclosure.

What is claimed is:

1. A method of inhibiting growth of cancer cells comprising inhibiting the expression or activity of ATF7IP and/or SETDB1 in the cancer cells.

2. The method of claim 1, wherein the expression or activity of ATF7IP and/or SETDB1 is inhibited by disrupting expression of the ATF7IP and/or SETDB1 using a CRISPR system and one or more guide RNAs targeted to the ATF7IP and/or the SETDB1.

3. The method of claim 1, wherein the expression or activity of ATF7IP and/or SETDB1 is inhibited by RNAi.

4. The method of claim 1, wherein the expression or activity of SETDB1 is inhibited in the cancer cells.

5. The method of claim 4, wherein the expression of SETDB1 is inhibited in the cancer cells.

6. The method of claim 1, wherein the expression or activity of ATF7IP is inhibited in the cancer cells.

7. The method of claim 6, wherein the expression of ATF7IP is inhibited in the cancer cells.

8. A method of identifying if an individual is suited for immune therapy comprising determining the expression or activity of ATF7IP and/or SETDB1 in a tumor sample obtained from the individual, and if the level is the same or lower that the level from a control sample, then identifying the individual to be suitable for immune therapy, and if the level is higher than the level from the control sample, then identifying the individual to be not suitable for immune therapy.

9. The method of claim 8, further comprising reducing the expression or activity of ATF7IP and/or SETDB1 in individuals identified as having higher than control levels of expression of ATF7IP and/or SETDB1.

10. The method of claim 9, wherein the expression or activity of ATF7IP and/or SETDB1 is inhibited using a CRISPR system and one or more guide RNAs targeted to the ATF7IP and/or the SETDB1.

11. The method of claim 10, wherein the expression or activity of ATF7IP and/or SETDB1 is inhibited by RNAi.

12. The method of claim 9, wherein a reduction in expression or activity of ATF7IP and/or SETDB1 is measured as restoration of immune surveillance in tumor cells, upregulation of expression of endogenous retroviral antigens, activation of a type I interferon immune response, or a combination thereof.

13. The method of claim 9, further comprising administering immune therapy to the individual.

14. The method of claim 13, wherein the immune therapy comprises immune checkpoint inhibition.

15. The method of claim 14, wherein the immune therapy comprises administration of antibodies that bind with specificity to PD-1, PD-L1, CTLA-4, LAG-3, Tim-1, 41BB, OX40, or CD122.

16. The method claim 9, wherein the individual is afflicted with lung cancer, colorectal cancer, or melanoma.

17. The method of claim 14, wherein the individual is afflicted with lung cancer, colorectal cancer, or melanoma.

18. The method of claim 15, wherein the individual is afflicted with lung cancer, colorectal cancer, or melanoma.

19. The method of claim 9, wherein the expression of SETDB1 is inhibited.

20. The method of claim 9, wherein the expression of ATF7IP is inhibited.
